SEQUENCE LISTING (110 sequences)

SEQ ID NO: 1 - DNA, 354 bp, Homo sapiens
  CDS (1)..(354)
caggtgaccttgaaggagtctggtcctgcgctggtgaaacccacacagaccctcacactgacctgcaccttctctgacttctcactcaccactagtggaatgcgtgtcagctggatccgtcagcccccagggaaggccctggagtggcttgcacgcattgattgggatgatgataagttctacagcacctctctgaagaccaggctcaccatctccaaggacacctccaaaaaccaggtggtccttacactgaccaacatggaccctgtggacacagccacgtattactgtgcacgggatatttcgaaagcacatgactactggggccagggaaccctggtcactgtctcctca

SEQ ID NO: 2 - protein, 118 aa, Homo sapiens
QVTLKESGPALVKPTQTLTLTCTFSDFSLTTSGMRVSWIRQPPGKALEWLARIDWDDDKFYSTSLKTRLTISKDTSKNQVVLTLTNMDPVDTATYYCARDISKAHDYWGQGTLVTVSS

SEQ ID NO: 3 - DNA, 324 bp, Homo sapiens
  CDS (1)..(324)
gaaacgacactcacgcagtctccagccaccctgtctgtgtctctaggggacagagcctccctctcctgcagggccagtcagagtgtgggcaccaagttagcctggtaccagcacaaacctggccaggctcccaggctcctcttctatggtgcatccaccagggcccctagtgtcccggccaggttcagtggcagtgggtctgggacagaattcactctcaccatcagcagcctgcagtctgaagattttgcagtttattactgtcagcaatatataaactggcctcggacgttcggccaagggaccaaggtggaaatcaaaggc

SEQ ID NO: 4 - protein, 108 aa, Homo sapiens
ETTLTQSPATLSVSLGDRASLSCRASQSVGTKLAWYQHKPGQAPRLLFYGASTRAPSVPARFSGSGSGTEFTLTISSLQSEDFAVYYCQQYINWPRTFGQGTKVEIKG

SEQ ID NO: 5 - DNA, 390 bp, Homo sapiens
  CDS (1)..(390)
caggtgcagctacagcagtggggcgcgggaatattgaagccttcggagaccctgtctctaacctgcgctatctacggcacatatatcggtggttatcactggacgtggatccgacggcccccaggggggggactggagtggattggagaaatcgaaaatactggtaacatcaaatataatccgtccctcaggaatcgagtcaccatttcctcagacacgtccaaaagacaagtcttcttgagtctgacttctctgaccactgcggacacggctgtatattattgtgcgaagggggggggtaaagaggtcacggttctcggcctgcctgtggggtgggactactacggcatggacatatggggcccagggaccacggtcaccgtctcctca

SEQ ID NO: 6 - protein, 130 aa, Homo sapiens
QVQLQQWGAGILKPSETLSLTCAIYGTYIGGYHWTWIRRPPGGGLEWIGEIENTGNIKYNPSLRNRVTISSDTSKRQVFLSLTSLTTADTAVYYCAKGGGKEVTVLGLPVGWDYYGMDIWGPGTTVTVSS

SEQ ID NO: 7 - DNA, 324 bp, Homo sapiens
  CDS (1)..(324)
gacatccagatgacccagtctccatcctccctgtctgcatctgtgggagacagagtcaccatcacttgccgggcaagtcagacccttaccacctatttaaattggtatcaacagagaccagggaaagccccaaaactcctcatctatcttgcatccactttggacagtggggtcccatcaagattcagtggcagtgcatctggcacaactttcaccctcaccatcaccagtctacaacctgacgatgttgcaacttattactgtcaacagagttacaatattcctcggacgttcggccaggggaccaaggtggagatcaaaggc

SEQ ID NO: 8 - protein, 108 aa, Homo sapiens
DIQMTQSPSSLSASVGDRVTITCRASQTLTTYLNWYQQRPGKAPKLLIYLASTLDSGVPSRFSGSASGTTFTLTITSLQPDDVATYYCQQSYNIPRTFGQGTKVEIKG

SEQ ID NO: 9 - DNA, 354 bp, Homo sapiens
  CDS (1)..(354)
caggtgaccttgaaggagtctggtcctacgctggtgaaacccacacagaccctcacgctgacctgcaccttctctggattctcactcagcactagtggaatgggtgtgggctggatccgtcagcccccaggaaaggccctggagtggcttgcagtcatttattgggatgatgataagcgctacagcccatctctgaagaccaggctcaccatcaccaaggacacctccaaaaaccaggttgtccttacaatgaccaacatggaccctgtggacacagccacatattactgtgcacagcaacggtggtccaactttgactactggggccagggaatcctggtcaccgtctcctca

SEQ ID NO: 10 - protein, 118 aa, Homo sapiens
QVTLKESGPTLVKPTQTLTLTCTFSGFSLSTSGMGVGWIRQPPGKALEWLAVIYWDDDKRYSPSLKTRLTITKDTSKNQVVLTMTNMDPVDTATYYCAQQRWSNFDYWGQGILVTVSS

SEQ ID NO: 11 - DNA, 327 bp, Homo sapiens
  CDS (1)..(327)
gaaattgtattgacgcagtctccagccaccctgtctttgtctccaggggaaagagccaccctctcctgcagggccagtcagagtgttagcggcaacttagcctggtaccagcagaaacctggccaggctcccaggctcctcatctttgctacatccaccagggccactggtatcccagtcaggtttagtggccgtgggtctgggacagacttcactctcaccatcagcagactggagcctgaagattttgcagtgtattactgtcagcagtatggtggctcacctcggctcactttcggcggagggaccaaggtggaaatcaaaggc

SEQ ID NO: 12 - protein, 109 aa, Homo sapiens
EIVLTQSPATLSLSPGERATLSCRASQSVSGNLAWYQQKPGQAPRLLIFATSTRATGIPVRFSGRGSGTDFTLTISRLEPEDFAVYYCQQYGGSPRLTFGGGTKVEIKG

SEQ ID NO: 13 - DNA, 366 bp, Homo sapiens
  CDS (1)..(366)
caggtgcagctacagcagtggggcgcaggactgttgaagccttcggagaccctgtccctcacctgcgctgtttctggttatgggtccctcagtggttactactggagctggatccgccagcccccagggaaggggctggagtggattggggaaatcaatcgtggcggaagcaccaaatacaacccgtccctcaagagtcgaatcaccatatcagtagacacgtccaagaaccagttctccctgaatctgaactctgtgaccgccgcggacacggctgtgtattactgtgcgagaagtggcctgtatggcaccactccgtcaacctttgaccactggggccagggaaccctggtcaccgtctcctca

SEQ ID NO: 14 - protein, 122 aa, Homo sapiens
QVQLQQWGAGLLKPSETLSLTCAVSGYGSLSGYYWSWIRQPPGKGLEWIGEINRGGSTKYNPSLKSRITISVDTSKNQFSLNLNSVTAADTAVYYCARSGLYGTTPSTFDHWGQGTLVTVSS

SEQ ID NO: 15 - DNA, 336 bp, Homo sapiens
  CDS (1)..(336)
cagtctgtggtgacgcagccgccctcagtgtctgggaccccaggatcgtgggtcaccatctcctgcactgggaccaggtccaacatcggggcacctaagggtgtccactggtaccaacacgtcccagggacagcccctaaactcctcatctatgatagtaatagtcggtcctcgggggtccctgcccgcttctctgggtccaagtctggcacttcaggcagcctggccatcgctggactgcaacctgaggatgaggctgattattattgccagtctcttgataccaacttgaatgcttgggtgttcggcggggggaccaagctgaccgtcctaggc

SEQ ID NO: 16 - protein, 112 aa, Homo sapiens
QSVVTQPPSVSGTPGSWVTISCTGTRSNIGAPKGVHWYQHVPGTAPKLLIYDSNSRSSGVPARFSGSKSGTSGSLAIAGLQPEDEADYYCQSLDTNLNAWVFGGGTKLTVLG

SEQ ID NO: 17 - DNA, 366 bp, Homo sapiens
  CDS (1)..(366)
caggtgcagctgcagcagtcgggcccaggactggtgaaggcttcggagaccctgtccctcacctgcgctgtctctggttactccatcagcagcggttactactggggctggatccggcagcccccagggaagggactggagtggattggaaatatctatcatagtgggaacacctactacaacccgtccctcaagagtcgagttaccatttcacgagacacgtccaagaatcacttctccctgaagctgaagtctgtgaccgccgcagacacggccgtgtattactgtgcgagagccgaccccggctatatgatctttaactggttcgacccctggggccagggaaccctggtcaccgtctcctca

SEQ ID NO: 18 - protein, 122 aa, Homo sapiens
QVQLQQSGPGLVKASETLSLTCAVSGYSISSGYYWGWIRQPPGKGLEWIGNIYHSGNTYYNPSLKSRVTISRDTSKNHFSLKLKSVTAADTAVYYCARADPGYMIFNWFDPWGQGTLVTVSS

SEQ ID NO: 19 - DNA, 324 bp, Homo sapiens
  CDS (1)..(324)
aattttatgctgactcagccaccctcagcgtctgggacccccgggcagagggtcaccatctcttgttctggaagcagctccaacatcggaaataatgatgtatactggtaccagcaattcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgatattcggcggagggaccaagctgaccgtcctaggc

SEQ ID NO: 20 - protein, 108 aa, Homo sapiens
NFMLTQPPSASGTPGQRVTISCSGSSSNIGNNDVYWYQQFPGTAPKLLIYRNNQRPSGVPDRFSGSKSGTSASLAISGLRSEDEADYYCAAWDDSLIFGGGTKLTVLG

SEQ ID NO: 21 - DNA, 369 bp, Homo sapiens
  CDS (1)..(369)
caggtgcagctggtggagtctgggtctgaggtgaagaagcctggggcctcagtgaaggtctcctgtaaggcttctggatacaccttcaccgactactatatccactgggtgcgacaggcccctggacaagggcttgaatggatgggatggctcaaccccaatggtggaagtacaaactatccacagaagtttcagggctgcgtcaccatgacccgggacacggccaccagtacagccttcatggagctgagcggcctcaagtctgacgacacggccttatattactgtgcgagagcgataaaagccgattcgggcttctcctaccttctggacgtctggggccaagggaccacggtcaccgtctcctca

SEQ ID NO: 22 - protein, 123 aa, Homo sapiens
QVQLVESGSEVKKPGASVKVSCKASGYTFTDYYIHWVRQAPGQGLEWMGWLNPNGGSTNYPQKFQGCVTMTRDTATSTAFMELSGLKSDDTALYYCARAIKADSGFSYLLDVWGQGTTVTVSS

SEQ ID NO: 23 - DNA, 333 bp, Homo sapiens
  CDS (1)..(333)
cagcctgtgctgactcagccaccctcagcgtctgggacccccgggcagagggtcaccatctcttgttctggaagcagctctaacatcggaagtaattatgtatactggtaccagcagctcccaggaacggcccccaaactcctcatctataggattaatcagcggccctcaggggtccgtgaccgattctctggctccaagtctggcacctcagcctccctggccatcagtgggctccggtccgaggatgaggctgattattactgttcagcatgggatgacagcctgagtggttgggtgttcggcggagggaccaagctgaccgtcctaggc

SEQ ID NO: 24 - protein, 111 aa, Homo sapiens
QPVLTQPPSASGTPGQRVTISCSGSSSNIGSNYVYWYQQLPGTAPKLLIYRINQRPSGVRDRFSGSKSGTSASLAISGLRSEDEADYYCSAWDDSLSGWVFGGGTKLTVLG

SEQ ID NO: 25 - DNA, 360 bp, Homo sapiens
  CDS (1)..(360)
caggtgcagctgcaggagtccgggggaggcttagttcagcccggagggtccctgagactctcctgtacagcctcaggattcactttcagtaatcactggatgcactgggtccgccaaactccagggaaggggctggtgtgggtctcacgtatcagaggtgatgcgggcgacagaaactacgcggactctgtgaagggccgattcaccatctccagagacaactcccagaacaccctgtttctgcaaatgaacagtctgacagccgaggacacggctgtctattactgtgcaagagatgtggtccttggttcgggcagtactgaccagtggggccagggagccctggtcaccgtctcctca

SEQ ID NO: 26 - protein, 120 aa, Homo sapiens
QVQLQESGGGLVQPGGSLRLSCTASGFTFSNHWMHWVRQTPGKGLVWVSRIRGDAGDRNYADSVKGRFTISRDNSQNTLFLQMNSLTAEDTAVYYCARDVVLGSGSTDQWGQGALVTVSS

SEQ ID NO: 27 - DNA, 342 bp, Homo sapiens
  CDS (1)..(342)
gatattgtgatgacccagactccagactccctggctgtgtctctgggcgagagggccaccatcaactgcaagtccagccagagtgttttatacagctccaacagtaagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagcttattactgtcagcaatattctactactccgttcactttcggcggagggaccaagctggagatcaaaggc

SEQ ID NO: 28 - protein, 114 aa, Homo sapiens
DIVMTQTPDSLAVSLGERATINCKSSQSVLYSSNSKNYLAWYQQKPGQPPKLLIYWASTRESGVPDRFSGSGSGTDFTLTISSLQAEDVAAYYCQQYSTTPFTFGGGTKLEIKG

SEQ ID NO: 29 - DNA, 362 bp, Homo sapiens
  CDS (1)..(360)
caggtgcagctggtggagtccgggggaggcgtggtccagcctgggaggtccctgagcctctcctgtacaacctctggattcaccttcagtgactattacatgcactgggtccgccaggctccaggcaaggggctggagtgggtggcatttatttcatctgatggaggtaataagtattatttagactccgtgaagggccgattcaccgtctccagagacaattccaagaacacactatctctgcaaatgaacagcctgaagactgaggacacggctctgtattactgtgcgggggtgccagctgttacgtcagcctggtacttcgatctctggggccgtggcaccctggtcaccgtctctca

SEQ ID NO: 30 - protein, 120 aa, Homo sapiens
QVQLVESGGGVVQPGRSLSLSCTTSGFTFSDYYMHWVRQAPGKGLEWVAFISSDGGNKYYLDSVKGRFTVSRDNSKNTLSLQMNSLKTEDTALYYCAGVPAVTSAWYFDLWGRGTLVTVS

SEQ ID NO: 31 - DNA, 342 bp, Homo sapiens
  CDS (1)..(342)
gacatccagttgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgagaatctggggtccctgaccggttcagtggcagcgggtctgggacagatttcactctcaccatcagcagcctgcaggttgaagatgtggcagtgtattattgtcagcaatattatacttattccctcactttcggcggagggaccaagctggagatcaaaggc

SEQ ID NO: 32 - protein, 114 aa, Homo sapiens
DIQLTQSPDSLAVSLGERATINCKSSQSVLYSSNNKNYLAWYQQKPGQPPKLLIYWASTRESGVPDRFSGSGSGTDFTLTISSLQVEDVAVYYCQQYYTYSLTFGGGTKLEIKG

SEQ ID NO: 33 - DNA, 357 bp, Homo sapiens
  CDS (1)..(357)
caggtgaccttgaaggagtctggtcctgcgctggtgaaatccacacagaccctcacgctgacctgcaacttgtctggtttctctgttaacactagaggagtgggtgtgggctggctccgtcagcccccaggaaaggccctggagtggcttgctatgatttattgggatgatgataggcgctacagtccatacgtgaaagacaggatctccctcaccagggacatctccaaaacccaggtggtccttacaatgaccaacatggagcctgccgacacaggcacatattactgtgcacgcgactacaatcctcggtcttttgatcactggggccagggaaccctggtcaccgtctcctca

SEQ ID NO: 34 - protein, 119 aa, Homo sapiens
QVTLKESGPALVKSTQTLTLTCNLSGFSVNTRGVGVGWLRQPPGKALEWLAMIYWDDDRRYSPYVKDRISLTRDISKTQVVLTMTNMEPADTGTYYCARDYNPRSFDHWGQGTLVTVSS

SEQ ID NO: 35 - DNA, 336 bp, Homo sapiens
  CDS (1)..(336)
aattttatgctgactcagcccccctcggcgtctgggacccccgggcagacggtctccatctcttgttctggaagcgactccaacatcggaacttatactgttaactggtaccagcaactcccaggaacggcccccaaactcctcatctatagcaatgatcagcggccctcaggggtccctgaccgattctctggctccaagtctggcacctcagcctccctggccatcagtgggctgcagtccgaggatgaggctgattatcactgtgcagcatgggatgccaatttgtatggccatgtggtattcggcggagggaccaaggtggaaatcaaaggc

SEQ ID NO: 36 - protein, 112 aa, Homo sapiens
NFMLTQPPSASGTPGQTVSISCSGSDSNIGTYTVNWYQQLPGTAPKLLIYSNDQRPSGVPDRFSGSKSGTSASLAISGLQSEDEADYHCAAWDANLYGHVVFGGGTKVEIKG

SEQ ID NO: 37 - DNA, 357 bp, Homo sapiens
  CDS (1)..(357)
caggtgcagctggtggagactgggggaggcttggtccagcctggggggtccctgagactctcctgtgcagcctcaggactcagctttagtaactatgcgatgagctgggtccgccaggctccaggaaaggggctggagtgggtctcaactatcagtaatagtggtgataacacactctacccagactccgtgaagggccggttcaccatctccagagacaattccagagacacactatatctgcacttgagtggcctgggagccgaggacacggccatttactactgtgcgaacgccccaccatacactcactactttgaccagtggggccagggaaccctggtcaccgtctcctca

SEQ ID NO: 38 - protein, 119 aa, Homo sapiens
QVQLVETGGGLVQPGGSLRLSCAASGLSFSNYAMSWVRQAPGKGLEWVSTISNSGDNTLYPDSVKGRFTISRDNSRDTLYLHLSGLGAEDTAIYYCANAPPYTHYFDQWGQGTLVTVSS

SEQ ID NO: 39 - DNA, 342 bp, Homo sapiens
  CDS (1)..(342)
gacatccggttgacccagtctccagactccctggctgtgtctctgggcgagagggccaccatcaactgcaagtccagtcagagtattttatacagatacaacagtaacaactacttagcttggtaccagcagaaaccaggacagcctcctaagttgctcatttactgggcatctacccgggaatccggggtccctgaccgattcagtggcagcgggtctgggacagatttcactctcaccatcagcagtctgcaggctgaagatgtggcagtttattactgtcagcaatattacagtactcctcagacgttcggccaagggaccaaggtggaaatcaaaggc

SEQ ID NO: 40 - protein, 114 aa, Homo sapiens
DIRLTQSPDSLAVSLGERATINCKSSQSILYRYNSNNYLAWYQQKPGQPPKLLIYWASTRESGVPDRFSGSGSGTDFTLTISSLQAEDVAVYYCQQYYSTPQTFGQGTKVEIKG

SEQ ID NO: 41 - DNA, 366 bp, Homo sapiens
  CDS (1)..(366)
caggtgcagctggtgcagtctgggggaggcctggtcaagcctggggggtccctgagactctcctgtgcagccactgggttcagtttcagacattatggcatgaactgggtccgccaggccccagggaaggggctggagtgggtctcgtacattagtgatagtagcagccacatctactacgcagactccgtgaggggccgattcagcatctccagagacaacgccaaggactcactgtttctggaagtgaacagcctgagacccggggacacggctgcttattactgtgcgagagatacgggcacctatgcccgcatgtccggtatggacgtctggggccaagggaccacggtcaccgtctcctca

SEQ ID NO: 42 - protein, 122 aa, Homo sapiens
QVQLVQSGGGLVKPGGSLRLSCAATGFSFRHYGMNWVRQAPGKGLEWVSYISDSSSHIYYADSVRGRFSISRDNAKDSLFLEVNSLRPGDTAAYYCARDTGTYARMSGMDVWGQGTTVTVSS

SEQ ID NO: 43 - DNA, 321 bp, Homo sapiens
  CDS (1)..(321)
gatattgtgctgacgcagactccatcctccctgtctgcatctgtaggagacagagtcaccatcacttgccgggccagtcagagtattagtgactggttggcctggtatcagcagaaaccagggaaagcccctaagctcctgatctataaggcgtctagtttagaaagtggggtcccgtcaaggttcagcggcagtggatctgggacagagttcactctcaccatcagcagcctgcagcctgatgattttgcaacttattactgccaacaatataatagttacttcactttcggccctgggaccaaagtggatatcaaaggc

SEQ ID NO: 44 - protein, 107 aa, Homo sapiens
DIVLTQTPSSLSASVGDRVTITCRASQSISDWLAWYQQKPGKAPKLLIYKASSLESGVPSRFSGSGSGTEFTLTISSLQPDDFATYYCQQYNSYFTFGPGTKVDIKG

SEQ ID NO: 45 - DNA, 363 bp, Homo sapiens
  CDS (1)..(363)
caggtgcagctgcagcagtggggcgcagggctggtgaagccttcggagaccctgtccctcacctgcgctatctatggtgggtccttcagttcttactactggaactggatccgccagcccccagggaagggactggagtggattggggaaatcaatcatagtggaggcaccaactacaacccgtccctcaagagtcgagtcaccatatcacttgacatgtccaagaaccacttctccctgaacctgaactctgtgaccgccgcggacacggctgtctattattgtgcgagagatgctggtaaccgggggtctacggactggttcgacccctggggccagggaaccctggtcaccgtctcctca

SEQ ID NO: 46 - protein, 121 aa, Homo sapiens
QVQLQQWGAGLVKPSETLSLTCAIYGGSFSSYYWNWIRQPPGKGLEWIGEINHSGGTNYNPSLKSRVTISLDMSKNHFSLNLNSVTAADTAVYYCARDAGNRGSTDWFDPWGQGTLVTVSS

SEQ ID NO: 47 - DNA, 324 bp, Homo sapiens
  CDS (1)..(324)
gacatccggatgacccagtctccatcctccctgtctgcatctgtaggagacagagtcaccatcacttgccgggcaagtcagggcattagaaatgatttaggctggtatcagcagagaccagggaaagccccgaatctcctgatctatgctgcatccactttacaaagtggggtcccatcaagattcagcggcagtggatccggcacagatttcactctcaccatcagcagcctgcagcctgaagattctgcaacttattactgtctacaagattacgcttacccacggacgttcggccaagggaccaaggtggaaatcaaaggc

SEQ ID NO: 48 - protein, 108 aa, Homo sapiens
DIRMTQSPSSLSASVGDRVTITCRASQGIRNDLGWYQQRPGKAPNLLIYAASTLQSGVPSRFSGSGSGTDFTLTISSLQPEDSATYYCLQDYAYPRTFGQGTKVEIKG

SEQ ID NO: 49 - DNA, 37 bp, Artificial Sequence
  Description of Artificial Sequence Synthetic primer
agcgcggcctctagacaggtgcagctgcaggagtcsg

SEQ ID NO: 50 - DNA, 36 bp, Artificial Sequence
  Description of Artificial Sequence Synthetic primer
agcgcggcctctagacaggtacagctgcagcagtca

SEQ ID NO: 51 - DNA, 37 bp, Artificial Sequence
  Description of Artificial Sequence Synthetic primer
agcgcggcctctagacaggtgcagctacagcagtggg

SEQ ID NO: 52 - DNA, 36 bp, Artificial Sequence
  Description of Artificial Sequence Synthetic primer
agcgcggcctctagagaggtgcagctgktggagwcy

SEQ ID NO: 53 - DNA, 38 bp, Artificial Sequence
  Description of Artificial Sequence Synthetic primer
agcgcggcctctagacaggtccagctkgtrgagtctgg

SEQ ID NO: 54 - DNA, 37 bp, Artificial Sequence
  Description of Artificial Sequence Synthetic primer
agcgcggcctctagacagrtcaccttgaaggagtctg

SEQ ID NO: 55 - DNA, 38 bp, Artificial Sequence
  Description of Artificial Sequence Synthetic primer
agcgcggcctctagacaggtgcagctggtgsartctgg

SEQ ID NO: 56 - DNA, 23 bp, Artificial Sequence
  Description of Artificial Sequence Synthetic primer
gacatccrgdtgacccagtctcc

SEQ ID NO: 57 - DNA, 23 bp, Artificial Sequence
  Description of Artificial Sequence Synthetic primer
gaaattgtrwtgacrcagtctcc

SEQ ID NO: 58 - DNA, 23 bp, Artificial Sequence
  Description of Artificial Sequence Synthetic primer
gatattgtgmtgacbcagwctcc

SEQ ID NO: 59 - DNA, 22 bp, Artificial Sequence
  Description of Artificial Sequence Synthetic primer
gaaacgacactcacgcagtctc

SEQ ID NO: 60 - DNA, 23 bp, Artificial Sequence
  Description of Artificial Sequence Synthetic primer
cagtctgtsbtgacgcagccgcc

SEQ ID NO: 61 - DNA, 22 bp, Artificial Sequence
  Description of Artificial Sequence Synthetic primer
tcctatgwgctgacwcagccac

SEQ ID NO: 62 - DNA, 23 bp, Artificial Sequence
  Description of Artificial Sequence Synthetic primer
tcctatgagctgayrcagcyacc

SEQ ID NO: 63 - DNA, 20 bp, Artificial Sequence
  Description of Artificial Sequence Synthetic primer
cagcctgtgctgactcaryc

SEQ ID NO: 64 - DNA, 23 bp, Artificial Sequence
  Description of Artificial Sequence Synthetic primer
cagdctgtggtgacycaggagcc

SEQ ID NO: 65 - DNA, 23 bp, Artificial Sequence
  Description of Artificial Sequence Synthetic primer
cagccwgkgctgactcagccmcc

SEQ ID NO: 66 - DNA, 23 bp, Artificial Sequence
  Description of Artificial Sequence Synthetic primer
tcctctgagctgastcaggascc

SEQ ID NO: 67 - DNA, 21 bp, Artificial Sequence
  Description of Artificial Sequence Synthetic primer
cagtctgyyctgaytcagcct

SEQ ID NO: 68 - DNA, 22 bp, Artificial Sequence
  Description of Artificial Sequence Synthetic primer
aattttatgctgactcagcccc

SEQ ID NO: 69 - DNA, 25 bp, Artificial Sequence
  Description of Artificial Sequence Synthetic primer
cattgaggagacrgtgaccagggtg

SEQ ID NO: 70 - DNA, 25 bp, Artificial Sequence
  Description of Artificial Sequence Synthetic primer
cattgaggagacggtgaccagggtt

SEQ ID NO: 71 - DNA, 24 bp, Artificial Sequence
  Description of Artificial Sequence Synthetic primer
cattgaagagacggtgaccattgt

SEQ ID NO: 72 - DNA, 26 bp, Artificial Sequence
  Description of Artificial Sequence Synthetic primer
cattgaggagacggtgaccgtggtcc

SEQ ID NO: 73 - DNA, 23 bp, Artificial Sequence
  Description of Artificial Sequence Synthetic primer
catggttggggcggatgcactcc

SEQ ID NO: 74 - DNA, 24 bp, Artificial Sequence
  Description of Artificial Sequence Synthetic primer
catsgatgggcccttggtggargc

SEQ ID NO: 75 - DNA, 20 bp, Artificial Sequence
  Description of Artificial Sequence Synthetic primer
tttgatttccaccttggtcc

SEQ ID NO: 76 - DNA, 20 bp, Artificial Sequence
  Description of Artificial Sequence Synthetic primer
tttgatctccascttggtcc

SEQ ID NO: 77 - DNA, 19 bp, Artificial Sequence
  Description of Artificial Sequence Synthetic primer
ttgatatccactttggtcc

SEQ ID NO: 78 - DNA, 20 bp, Artificial Sequence
  Description of Artificial Sequence Synthetic primer
tttaatctccagtcgtgtcc

SEQ ID NO: 79 - DNA, 20 bp, Artificial Sequence
  Description of Artificial Sequence Synthetic primer
taggacggtsascttggtcc

SEQ ID NO: 80 - DNA, 20 bp, Artificial Sequence
  Description of Artificial Sequence Synthetic primer
gaggacggtcagctgggtgc

SEQ ID NO: 81 - DNA, 22 bp, Artificial Sequence
  Description of Artificial Sequence Synthetic primer
ggacacggccgtrtattactgt

SEQ ID NO: 82 - DNA, 22 bp, Artificial Sequence
  Description of Artificial Sequence Synthetic primer
ggacayggccatgtattactgt

SEQ ID NO: 83 - DNA, 24 bp, Artificial Sequence
  Description of Artificial Sequence Synthetic primer
atcagywgnstgsarsctgargat

SEQ ID NO: 84 - DNA, 24 bp, Artificial Sequence
  Description of Artificial Sequence Synthetic primer
atcaatwgnstgsarsctgargat

SEQ ID NO: 85 - DNA, 21 bp, Artificial Sequence
  Description of Artificial Sequence Synthetic primer
gaygaggctgactattactgt

SEQ ID NO: 86 - DNA, 21 bp, Artificial Sequence
  Description of Artificial Sequence Synthetic primer
gatgaatctgattattactgt

SEQ ID NO: 87 - DNA, 21 bp, Artificial Sequence
  Description of Artificial Sequence Synthetic primer
gatgaggctgagtatcattgt

SEQ ID NO: 88 - DNA, 21 bp, Artificial Sequence
  Description of Artificial Sequence Synthetic primer
gatgagagtgactaccactgt

SEQ ID NO: 89 - DNA, 22 bp, Artificial Sequence
  Description of Artificial Sequence Synthetic primer
acagtaatayacggccgtgtcc

SEQ ID NO: 90 - DNA, 22 bp, Artificial Sequence
  Description of Artificial Sequence Synthetic primer
acagtaatacatggccrtgtcc

SEQ ID NO: 91 - DNA, 24 bp, Artificial Sequence
  Description of Artificial Sequence Synthetic primer
atcytcagsytscasncwrctgat

SEQ ID NO: 92 - DNA, 24 bp, Artificial Sequence
  Description of Artificial Sequence Synthetic primer
atcytcagsytscasncwattgat

SEQ ID NO: 93 - DNA, 21 bp, Artificial Sequence
  Description of Artificial Sequence Synthetic primer
acagtaatagtcagcctcrtc

SEQ ID NO: 94 - DNA, 21 bp, Artificial Sequence
  Description of Artificial Sequence Synthetic primer
acagtaataatcagattcatc

SEQ ID NO: 95 - DNA, 21 bp, Artificial Sequence
  Description of Artificial Sequence Synthetic primer
acaatgatactcagcctcatc

SEQ ID NO: 96 - DNA, 21 bp, Artificial Sequence
  Description of Artificial Sequence Synthetic primer
acagtggtagtcactctcatc

SEQ ID NO: 97 - DNA, 29 bp, Artificial Sequence
  Description of Artificial Sequence Synthetic primer
tgtgatggcttccatgtcggcagaatgct

SEQ ID NO: 98 - DNA, 31 bp, Artificial Sequence
  Description of Artificial Sequence Synthetic primer
gctaaacaactttcaacagtctatgcggcac

SEQ ID NO: 99 - protein, 8 aa, Homo sapiens
DISKAHDY

SEQ ID NO: 100 - protein, 22 aa, Homo sapiens
GGGKEVTVLGLPVGWDYYGMDI

SEQ ID NO: 101 - protein, 8 aa, Homo sapiens
QRWSNFDY

SEQ ID NO: 102 - protein, 13 aa, Homo sapiens
SGLYGTTPSTFDH

SEQ ID NO: 103 - protein, 13 aa, Homo sapiens
ADPGYMIFNWFDP

SEQ ID NO: 104 - protein, 13 aa, Homo sapiens
AIKADSGFYLLDV

SEQ ID NO: 105 - protein, 11 aa, Homo sapiens
DVVLGSGSTDQ

SEQ ID NO: 106 - protein, 21 aa, Homo sapiens
QFQEHTISANEQPEDGHGSVL

SEQ ID NO: 107 - protein, 9 aa, Homo sapiens
DYNPRSFDH

SEQ ID NO: 108 - protein, 11 aa, Homo sapiens
NAPPYTHYFDQ

SEQ ID NO: 109 - protein, 13 aa, Homo sapiens
DTGTYARMSGMDV

SEQ ID NO: 110 - protein, 13 aa, Homo sapiens
DAGNRGSYDWFDP

(19) United States
(12) Patent Application Publication
(10) Pub. No.: US 2010/0324271 A1
(43) Pub. Date: Dec. 23, 2010

(54) PHAGE-DISPLAYING SINGLE-CHAIN ANTIBODY CAPABLE OF RECOGNIZING NON-REDUCED MANNOSE RESIDUE

(75) Inventors: Yoko Yamaguchi, Kanagawa (JP); Munehiro Nakata, Kanagawa (JP); Keiko Sakai, Kanagawa (JP); Yoshitaka Shimizu, Kanagawa (JP); Ayano Takasaki, Kanagawa (JP); Tomoki Chiba, Kanagawa (JP); Yu Kusada, Kanagawa (JP); Nobuyoshi Shimizu, Tokyo (JP); Atsushi Takayanagi, Tokyo (JP)

Correspondence Address: GREENBLUM & BERNSTEIN, P.L.C., 1950 ROLAND CLARKE PLACE, RESTON, VA, 20191, US

(73) Assignee: TOKAI UNIVERSITY EDUCATIONAL SYSTEM, Tokyo (JP)

(21) Appl. No.: 12/300,874

(22) PCT Filed: May 17, 2007
(86) PCT No.: PCT/JP2007/060146

§ 371 Date: Apr. 15, 2009

(30) Foreign Application Priority Data
May 17, 2006 (JP) 2006-137604

(51) Int. Cl. C07K 16/44; C07H 21/04

(52) U.S. Cl. 530/387.3; 536/23.53

(57) ABSTRACT

It is an object of the present invention to provide antibodies which recognize non-reducing mannose residues. The present invention provides antibodies recognizing non-reducing mannose residues, which are obtained through screening of phage-display library of human scFvs by using, as an antigen, Man3-DPPE which is an artificial glycolipid synthesized from mannotriose and dipalmitoylphosphatidylethanolamine by reductive amination.

### TECHNICAL FIELD

The present invention relates to phage-displayed single chain antibodies which recognize non-reducing mannose residues. More specifically, the present invention relates to single chain antibodies recognizing non-reducing mannose residues which are obtained by screening of phage-display antibody library.

### BACKGROUND ART

As generally known, immunization with carbohydrates often leads to a primary IgM response and no response in some cases because many carbohydrates are self-antigens (Non-patent Documents 1 and 2). Phage display technology, which allows generation of antibodies against self-antigens, is thus considered the best available strategy to produce antibodies directed against carbohydrate moieties. The possibility of isolating antibody fragments with high specificity and affinity for carbohydrates by phage display technique is appealing since conventional hybridoma technologies have proven to be ineffective in producing monoclonal antibodies directed against a variety of carbohydrate moieties. Phage display technology has been used mostly to generate antibodies against proteins, whereas its use for carbohydrate antigens has been rarely done. Because of difficulties in immobilization of carbohydrate antigens onto plastic plates, the same procedures generally used for protein antigens cannot be directly applied to carbohydrate antigens. Previous studies thus utilized glycoproteins, heteroglycans, and carbohydrate-BSA conjugates as antigens to produce anti-carbohydrate single chain antibodies (scFvs) by phage display methods (Non-patent Documents 1, and 3 to 7). Since the majority of anti-carbohydrate antibodies generated so far belong to the IgM class if they are monoclonal antibodies, or have low affinity if they are scFvs, they are not suitable for in vivo diagnostics or therapy. Low affinity to carbohydrate antigens is most likely due to the intrinsic nature of carbohydrates, which may not be easily overcome. Attempts have been made to overcome the low-affinity nature of anti-carbohydrate antibodies by increasing the antibody titer (Non-patent Documents 7 to 10). It has been shown that multivalent scFv have higher affinity to carbohydrate moieties (Non-patent Documents 9 to 10).
[Non-patent Document 1] Deng, S., MacKenzie, C. R., Sadowaka, J., Michniewicz, J., Young, N. M., Bundle, D. R., and Narang, S. A. (1994) J. Biol. Chem. 269, 9533-9538.
[Non-patent Document 2] MacKenzie, C. R., Hirama. T., Deng, S., Bundle, D. R., Narang, S. A., and Young, N. M. (1996) J. Biol. Chem. 271, 1527-1533.
[Non-patent Document 3] Babino, A., Pritsch, O., Oppezzo, P., Du Pasquier, R., Roseto, A., Osinaga, E., and Alzari, P. M. (1997) Hybridoma 16, 317-324.
[Non-patent Document 4] van Kuppevelt, T. H., Dennissen, M. A. B. A., van Venrooij, W. J., Hoet, R. M. A., and Veerkamp, J. H. (1998) J. Biol. Chem. 273, 12960-12966.
[Non-patent Document 5] Mao, S., Gao, C., Lo, C.-H. L., Wirsching, P., Wong, C.-H., and Janda K. D. (1999) Proc. Natl. Acad. Sci. USA 96, 6953-6958.
[Non-patent Document 6] Lee, K. J., Mao. S., Sun, C., Gao, C., Blixt, O., Arrues, S., Horn. L. G., Kaufmann, G. F., Hoffman, T. Z., Coyle, A. R., Paulson, J., Felding-Habermann, B., and Janda, K. D. (2002) J. Am. Chem. Soc. 124, 12439-12446.
[Non-patent Document 7] Ravn, P., Danielczyk, A., Jensen, K. B., Kristensen, P., Christensen, P. A., Larsen, M., Karsten, U., and Goletz, S. (2004) J. Mol. Biol. 343, 985-996
[Non-patent Document 8] Deng, S., MacKenzie, C. R., Hirama, T., Brousseau, R., Lowary, T. L., Young, N. M., Bundle, D. R., and Narang, S. A. (1995) Proc. Natl. Acad. Sci. USA 92, 4992-4996.
[Non-patent Document 9] MacKenzie, R. and To, R. (1998) J. Immunol. Methods 220, 39-49.
[Non-patent Document 10] Zhang, J., Tanha, J., Hirama, T., To, N. H. K. R., Tong-Sevinc, H., Stone, E., Brisson, J.-R., and MacKenzie, C. R. (2004) J. Mol. Biol. 345, 49-56.

### DISCLOSURE OF THE INVENTION

### Object to be Solved by the Invention

It is an object of the present invention to provide antibodies which recognize non-reducing mannose residues, and nucleic acids encoding the antibodies.

### Means for Solving the Object

The inventors of the present invention have adapted phage-display technology to generate human scFvs using artificial glycolipids as antigens (Stoll, M. S., Mizuochi, T., Childs, R. A., and Feizi, T. (1988) J. Biochem. 256, 661-665; Kenjo, A., Takahashi, M., Matsushita, M., Endo, Y., Nakata, M., Mizuochi, T., and Fujita, T. (2001) J. Biol. Chem. 276, 19959-19965; and Shimizu, Y., Nakata, M., Matsunuma, J., and Mizuochi, T. (2001) J. Chromatogr. B. Biomed. Sci. Appl. 754, 127-133). It is possible to isolate candidate phage antibodies directed against various carbohydrate antigens by immobilizing soluble carbohydrate antigens via lipid anchors on plastic titer-plates, and subsequent panning and screening by ELISA. To achieve the above object, 30 or more types of artificial glycolipids were synthesized in this invention.

To establish methodologies to build up a set of scFvs directed against various carbohydrate moieties, the inventors of the present invention have screened phage-display library representing 10¹¹ or more types of human scFvs (Sblattero, D. and Bradbury, A. (2000) Nat. Biotechnol. 18, 76-80). The present invention used, as a model antigens, Man3-DPPE which is an artificial glycolipid synthesized from mannotriose (Man3) and dipalmitoylphosphatidylethanolamine (DPPE) by reductive amination according to previous reports (Stoll, M. S., Mizuochi, T., Childs, R. A., and Feizi, T. (1988) J. Biochem. 256, 661-665; Kenjo, A., Takahashi, M., Matsushita, M., Endo, Y., Nakata, M., Mizuochi, T., and Fujita, T. (2001) J. Biol. Chem. 276, 19959-19965; and Shimizu, Y., Nakata, M., Matsunuma, J., and Mizuochi, T. (2001) J. Chromatogr. B. Biomed. Sci. Appl. 754, 127-133). Characterization of positive phage clones by TLC-overlay assays and surface plasmon resonance analyses revealed that these phage antibody clones bound to artificial glycolipids bearing mannose residues at nonreducing termini. In addition, binding of the phage antibodies to RNase B carrying high mannose type oligosaccharides but not to fetuin carrying complex type and O-linked oligosaccharides was observed. Furthermore, first round characterization of scFvs produced from respective phages indicated good affinity and specificity for nonreducing mannose residues. Thus, it was demonstrated the validity of a phage display technology in constructing human scFv against various carbohydrate antigens.

Thus the present invention provides an antibody of any of the following (1) to (14):

### (1) An Antibody Recognizing a Non-Reducing Mannose Residue, Comprising

the amino acid sequence of SEQ ID NO: 2 or an amino acid sequence, wherein one or a few amino acids have been deleted, substituted, or added in the amino acid sequence of SEQ ID NO: 2, as an amino acid sequence of heavy chain variable region, and the amino acid sequence of SEQ ID NO: 4 or an amino acid sequence, wherein one or a few amino acids have been deleted, substituted, or added in the amino acid sequence of SEQ ID NO: 4, as an amino acid sequence of light chain variable region;

### (2) An Antibody Recognizing a Non-Reducing Mannose Residue, Comprising

the amino acid sequence of SEQ ID NO: 6 or an amino acid sequence, wherein one or a few amino acids have been deleted, substituted, or added in the amino acid sequence of SEQ ID NO: 6, as an amino acid sequence of heavy chain variable region, and the amino acid sequence of SEQ ID NO: 8 or an amino acid sequence, wherein one or a few amino acids have been deleted, substituted, or added in the amino acid sequence of SEQ ID NO: 8, as an amino acid sequence of light chain variable region;

### (3) An Antibody Recognizing a Non-Reducing Mannose Residue, Comprising

the amino acid sequence of SEQ ID NO: 10 or an amino acid sequence, wherein one or a few amino acids have been deleted, substituted, or added in the amino acid sequence of SEQ ID NO: 10, as an amino acid sequence of heavy chain variable region, and the amino acid sequence of SEQ ID NO: 12 or an amino acid sequence, wherein one or a few amino acids have been deleted, substituted, or added in the amino acid sequence of SEQ ID NO: 12, as an amino acid sequence of light chain variable region;

### (4) An Antibody Recognizing a Non-Reducing Mannose Residue, Comprising

the amino acid sequence of SEQ ID NO: 14 or an amino acid sequence, wherein one or a few amino acids have been deleted, substituted, or added in the amino acid sequence of SEQ ID NO: 14, as an amino acid sequence of heavy chain variable region, and the amino acid sequence of SEQ ID NO: 16 or an amino acid sequence, wherein one or a few amino acids have been deleted, substituted, or added in the amino acid sequence of SEQ ID NO: 16, as an amino acid sequence of light chain variable region;

### (5) An Antibody Recognizing a Non-Reducing Mannose Residue, Comprising

the amino acid sequence of SEQ ID NO: 18 or an amino acid sequence, wherein one or a few amino acids have been deleted, substituted, or added in the amino acid sequence of SEQ ID NO: 18, as an amino acid sequence of heavy chain variable region, and the amino acid sequence of SEQ ID NO: 20 or an amino acid sequence, wherein one or a few amino acids have been deleted, substituted, or added in the amino acid sequence of SEQ ID NO: 20, as an amino acid sequence of light chain variable region;

### (6) An Antibody Recognizing a Non-Reducing Mannose Residue, Comprising

the amino acid sequence of SEQ ID NO: 22 or an amino acid sequence, wherein one or a few amino acids have been deleted, substituted, or added in the amino acid sequence of SEQ ID NO: 22, as an amino acid sequence of heavy chain variable region, and the amino acid sequence of SEQ ID NO: 24 or an amino acid sequence, wherein one or a few amino acids have been deleted, substituted, or added in the amino acid sequence of SEQ ID NO: 24, as an amino acid sequence of light chain variable region;

### (7) An Antibody Recognizing a Non-Reducing Mannose Residue, Comprising

the amino acid sequence of SEQ ID NO: 26 or an amino acid sequence, wherein one or a few amino acids have been deleted, substituted, or added in the amino acid sequence of SEQ ID NO: 26, as an amino acid sequence of heavy chain variable region, and the amino acid sequence of SEQ ID NO: 28 or an amino acid sequence, wherein one or a few amino acids have been deleted, substituted, or added in the amino acid sequence of SEQ ID NO: 28, as an amino acid sequence of light chain variable region;

### (8) An Antibody Recognizing a Non-Reducing Mannose Residue, Comprising

the amino acid sequence of SEQ ID NO: 30 or an amino acid sequence, wherein one or a few amino acids have been deleted, substituted, or added in the amino acid sequence of SEQ ID NO: 30, as an amino acid sequence of heavy chain variable region, and the amino acid sequence of SEQ ID NO: 32 or an amino acid sequence, wherein one or a few amino acids have been deleted, substituted, or added in the amino acid sequence of SEQ ID NO: 32, as an amino acid sequence of light chain variable region;

### (9) An Antibody Recognizing a Non-Reducing Mannose Residue, Comprising

the amino acid sequence of SEQ ID NO: 34 or an amino acid sequence, wherein one or a few amino acids have been deleted, substituted, or added in the amino acid sequence of SEQ ID NO: 34, as an amino acid sequence of heavy chain variable region, and the amino acid sequence of SEQ ID NO: 36 or an amino acid sequence, wherein one or a few amino acids have been deleted, substituted, or added in the amino acid sequence of SEQ ID NO: 36, as an amino acid sequence of light chain variable region;

### (10) An Antibody Recognizing a Non-Reducing Mannose Residue, Comprising

the amino acid sequence of SEQ ID NO: 38 or an amino acid sequence, wherein one or a few amino acids have been deleted, substituted, or added in the amino acid sequence of SEQ ID NO: 38, as an amino acid sequence of heavy chain variable region, and the amino acid sequence of SEQ ID NO: 40 or an amino acid sequence, wherein one or a few amino acids have been deleted, substituted, or added in the amino acid sequence of SEQ ID: 40, as an amino acid sequence of light chain variable region;

### (11) An Antibody Recognizing a Non-Reducing Mannose Residue, Comprising

the amino acid sequence of SEQ ID NO: 42 or an amino acid sequence, wherein one or a few amino acids have been deleted, substituted, or added in the amino acid sequence of SEQ ID NO: 42, as an amino acid sequence of heavy chain variable region, and the amino acid sequence of SEQ ID NO: 44 or an amino acid sequence, wherein one or a few amino acids have been deleted, substituted, or added in the amino acid sequence of SEQ ID: 44, as an amino acid sequence of light chain variable region; and

### (12) An Antibody Recognizing a Non-Reducing Mannose Residue, Comprising

the amino acid sequence of SEQ ID NO: 46 or an amino acid sequence, wherein one or a few amino acids have been deleted, substituted, or added in the amino acid sequence of SEQ ID NO: 46, as an amino acid sequence of heavy chain variable region, and the amino acid sequence of SEQ ID NO: 48 or an amino acid sequence, wherein one or a few amino acids have been deleted, substituted, or added in the amino acid sequence of SEQ ID NO: 48, as an amino acid sequence of light chain variable region.

Another aspect of the present invention provides a nucleic acid encoding the aforementioned antibody of the present invention.

Further another aspect of the present invention provides a nucleic acid of any of the following:
(1) a nucleic acid comprising the nucleotide sequence of SEQ ID NO: 1 and the nucleotide sequence of SEQ ID NO: 3;
(2) a nucleic acid comprising the nucleotide sequence of SEQ ID NO: 5 and the nucleotide sequence of SEQ ID NO: 7;
(3) a nucleic acid comprising the nucleotide sequence of SEQ ID NO: 9 and the nucleotide sequence of SEQ ID NO: 11;
(4) a nucleic acid comprising the nucleotide sequence of SEQ ID NO: 13 and the nucleotide sequence of SEQ ID NO: 15;
(5) a nucleic acid comprising the nucleotide sequence of SEQ ID NO: 17 and the nucleotide sequence of SEQ ID NO: 19;
(6) a nucleic acid comprising the nucleotide sequence of SEQ ID NO: 21 and the nucleotide sequence of SEQ ID NO: 23;
(7) a nucleic acid comprising the nucleotide sequence of SEQ ID NO: 25 and the nucleotide sequence of SEQ ID NO: 27;
(8) a nucleic acid comprising the nucleotide sequence of SEQ ID NO: 29 and the nucleotide sequence of SEQ ID NO: 31;
(9) a nucleic acid comprising the nucleotide sequence of SEQ ID NO: 33 and the nucleotide sequence of SEQ ID NO: 35;
(10) a nucleic acid comprising the nucleotide sequence of SEQ ID NO: 37 and the nucleotide sequence of SEQ ID NO: 39;
(11) a nucleic acid comprising the nucleotide sequence of SEQ ID NO: 41 and the nucleotide sequence of SEQ ID NO: 43; and
(12) a nucleic acid comprising the nucleotide sequence of SEQ ID NO: 45 and the nucleotide sequence of SEQ ID NO: 47.

Further another aspect of the present invention provides a process for producing an antibody wherein the aforementioned nucleic acid of the present invention is used.

Further another aspect of the present invention provides a recombinant antibody recognizing a nonreducing mannose residue which can be obtained by the aforementioned process. Preferably, the antibody is a single chain antibody.

### BEST MODE FOR CARRYING OUT THE INVENTION

Hereafter, embodiments of the present invention will be more specifically described.

The antibodies of the present invention are antibodies which recognize non-reducing mannose residues. As described in the Examples below, the antibodies of the present invention have been obtained through screening of phage-display library of human scFvs, with use of Man3-DPPE, as a model antigen, which is an artificial glycolipid synthesized from mannotriose (Man3) and dipalmitoylphosphatidylethanolamine (DPPE) by reductive amination. As a result of characterization of positive phage clones by TLC-overlay assays and surface plasmon resonance analyses, it was demonstrated that the antibodies of the present invention bound to artificial glycolipids bearing mannose residues at nonreducing termini.

The glycolipid for use as an antigen in screening of the present invention is not specifically limited, and any artificial glycolipid bearing a mannose residue at a nonreducing terminal may be used, although oligosaccharides are preferred.

In an oligosaccharide, individual constituent sugars bind to one another via an α→1,2 bond, an α→1,3 bond, an α→1,4 bond, an α→1,6 bond, β→1,4 bond, or a combination thereof. For example, mannose may constitute a straight chain through the abovementioned bond, or may adopt a branched structure via the combination of an α→1,3 bond with an α→1,6 bond. The number of monosaccharides in an oligosaccharide is preferably 2 to 11.

Specific examples of such an oligosaccharide include mannobiose (Man2), mannotriose (Man3), mannotetraose (Man4), mannopentaose (Man5), mannohexaose (Man6), and mannoheptaose (Man7).

These oligosaccharides have a reducing terminal aldehyde group. Thus, such an aldehyde group can be used as a means for immobilizing an oligosaccharide. That is to say, an aldehyde group is allowed to react with a lipid having an amino group to form a Schiff base. Subsequently, according to a common method, the Schiff base is reduced, and is preferably chemically reduced by NaBH₃CN, for example, so as to bind the oligosaccharide to the lipid.

The aforementioned lipid having an amino group is preferably a phospholipid having an amino group. For example, phosphatidylamine such as dipalmitoylphosphatidylethanolamine (DPPE) or distearoylphosphatidylethanolamine (DSPE) can be used. Thus obtained bound substance of an oligosaccharide and a lipid may be referred to as an artificial glycolipid in the present invention. The glycolipid for use in the present invention is preferably an artificial glycolipid as mentioned above.

The solvent for dissolving an artificial glycolipid serving as an antigen is not specifically limited, although it is preferable to dissolve such an artificial glycolipid in a solvent system of chloroform/methanol/water (10:10:3, v/v) and subsequently dilute it with methanol at 40 μg/ml, for example. This solution is added in, for example, a 96-well plastic plate (flat bottom) and dried in air so as to immobilize the artificial glycolipid onto the wells. These wells are blocked and washed. The plate coated with an artificial glycolipid by such an operation can be used as an antigen-coated plate.

Tris-buffered saline (hereunder, referred to as TBS) is desirably used as the buffer solution. In addition, for example, TBS containing 3% bovine serum albumin (hereunder, referred to as 3% BSA/TBS) is desirable as the blocking solution, and TBS containing 0.2% Tween 20 (hereunder, referred to as TBS-T) is desirable as the washing solution.

The phage-displayed antibodies can be produced by publicly known methods (Marks J D et al. (1991) J. Mol. Biol., 222, 581-597; Nissim A. et al. (1994) EMBO J., 13, 692-698; TAKAYANAGI Atsushi, OKUI Michiyo, and SHIMIZU, Nobuyoshi, ARTIFICIAL ANTIBODY LIBRARY WITH SUPER-REPERTORY, Japanese Patent Application No. 2001-358602 (PCT International Publication No. WO 03/04419)).

The phage-display antibody library is constructed by adapting a system in which antibodies are fused with the coat protein of a filamentous phage to thereby display these antibodies on the phage surface. Specifically, such a phage-display library can be made by PCR amplification of antibody genes to produce a library including many types of antibody genes, and displaying them on a phage. Hereunder, specific example of the method for producing phage-displayed single chain antibodies is described, but is not to be considered as limiting.

Human peripheral blood cDNA library and human spleen cDNA library are used as templates to amplify a fragment including CDR1 and CDR2 regions in VH or VL region of immunoglobulin gene, and a fragment including CDR3 region by PCR. These fragments are mixed and used as templates to amplify the VH or VL region by PCR. These amplified DNA fragments are respectively inserted into a non-expressing phagemid vector to produce a VH library and a VL library. These E. coli VH library and VL library are infected with helper phages to convert into phage libraries. These phage-type VH library and VL library are coinfected into E. coli expressing Cre-recombinase, to effect recombination between the VH and VL vectors within E. coli. This E. coli strain is infected with helper phages M13KO7, by which phages expressing full length single-chain antibodies can be produced. To remove unrecombinants mixed in these phages, E. coli is infected with these phages while avoiding multiple infection, then superinfected with helper phages, and incubated in a medium which contains ampicillin/kanamycin/chloramphenicol but does not contain glucose at 25° C. By so doing, phages expressing recombinant single-chain antibodies can be obtained in the supernatant. The phages in the supernatant are precipitated with polyethylene glycol and resuspended, by which the artificial antibody library with a large repertoire (10¹¹ or more clones) can be obtained.

Further, a phage-displayed antibody having a binding property to a carbohydrate of interest can be selectively collected from the human single-chain phage library produced by the above manner, with reference to the binding property to an artificial glycolipid having the concerned carbohydrate structure. This operation may be referred to as panning.

The carbohydrate-specific binding property of phage-displayed antibodies obtained by repetition of panning is desirably analyzed by the ELISA method that will be described later, for example, because a large number of phage clone samples can be simultaneously analyzed. That is to say, a carbohydrate is immobilized onto a 96-well plastic plate and blocked with a buffer solution containing 3% BSA for example, in order to avoid non-specific adsorption of phage-displayed antibodies. A suspension of the phage-displayed antibodies which had been adjusted at an appropriate concentration with a buffer solution is added thereto, followed by sufficient reaction (for example, at 37° C. for 2 hours), and subsequent washing with a buffer solution.

To detect/quantify phage-displayed antibodies bound to the carbohydrate, antibodies are reacted with an aqueous solution containing anti-phage antibodies that have been labeled with a substance suitable for detection, followed by washing with a buffer solution, and subsequent detection/quantification in an optimum method for detecting the labeling substance. At this time, there is no specific limitation in the labeling substance for the anti-phage antibodies, although labeling with horseradish peroxidase is desirable for example, in terms of handiness. In addition, there is no specific limitation in the detector for use in the detection, although a plate reader suitable for ELISA is desirably employed.

The term “antibody” used in the present invention does not only refer to antibodies in a form normally existing in vivo, but also include peptides containing at least one antigen-binding site formed of the variable region in the H chain or L chain of the antibody, or a combination thereof, Fab composed of a set of an H chain fragment and an L chain fragment, F(ab′)₂ composed of two sets of H chain fragments and L chain fragments, single chain antibodies composed of an H chain fragment and an L chain fragment bound in series in a single peptide (hereunder, may be referred to as “scFvs”), and the like. The “antibody” of the present invention may be a full length antibody in a form normally existing in vivo, which is composed of two sets of full length H chains and full length L chains.

The terms “F(ab′)₂” and “Fab” in the present invention mean antibody fragments produced by treatment of an immunoglobulin with a protease such as pepsin or papain, and generated by digestion around the disulfide bonds existing between two H chains in the hinge region. For example, when IgG1 is treated with papain, cleavages occur in upstreams of the disulfide bonds existing between two H chains in the hinge region to allow the production of two identical antibody fragments in which an L chain composed of VL (L chain variable region) and CL (L chain constant region) and an H chain fragment composed of VH (H chain variable region) and CHγ1 (γ1 region within H chain constant region) are connected by a disulfide bond at the C-terminal region. These two identical antibody fragments are respectively referred to as Fab′. In addition, when IgG is treated with pepsin, cleavages occur in downstreams of the disulfide bonds existing between two H chains in the hinge region to allow the production of an antibody fragment which is slightly larger than the combined product having said two Fab's connected by the hinge region. This antibody fragment is referred to as F(ab′)₂.

Usually, an antibody consists of two types of large and small polypeptides. The large subunit is referred to as “H chain (heavy chain)” and the small subunit is referred to as “L chain (light chain)”. In addition, each peptide is composed of a “variable region” existing at the N-terminal side and forming an antigen-binding site, and a “constant region” which is conserved per each antibody class. The variable region is further divided into complementarity determining regions “CDRs” which particularly involve the formation of the antigen-binding site, and “framework regions” existing therebetween. CDRs are known consist of three regions called “CDR1”, “CDR2”, and “CDR3” from the N-terminal side, for each H chain and L chain.

The single chain antibodies of the present invention can be prepared by appropriately selecting inducible vectors such as pSE380 plasmid (Invitrogen) or pET24d(+) plasmid (Novagen) and host bacterial cells. In addition to the above method, in the production of the antibodies of the present invention, an animal cell expression system, an insect cell expression system, and a yeast cell expression system can also be used. The linker for linking the H chain and the L chain can also be appropriately selected by those skilled in the art.

SEQ ID NOS: 1 to 48 in the Sequence Listing as shown below represent the nucleotide sequences and the amino acid sequences of H chains and L chains of the antibodies of the present invention. Amino acid sequences in which one or a few amino acids (the number of amino acids is preferably 1 to 8, more preferably 1 to 5, and yet more preferably 1 to 3) have been deleted, substituted, or added in the aforementioned amino acid sequences are also included in the scope of the present invention as long as an antibody recognizing a non-reducing mannose residue of a glycolipid can be provided. Nucleic acids comprising the nucleotide sequences represented in the Sequence Listing shown below can be obtained by either the procedure described in Examples of the present application, or chemical synthesis with use of a DNA synthesizer. In addition, nucleotide sequences encoding the amino acid sequences in which one or a few amino acids have been deleted, substituted, or added in the amino acid sequences in the Sequence Listing shown below, can also be readily obtained through usual gene recombination technique such as PCR, or chemical synthesis with use of a DNA synthesizer, by those skilled in the art.
SEQ ID NO:1:Nucleotide sequence of variable region of heavy chain of antibody M3-2
SEQ ID NO:2: Amino acid sequence of variable region of heavy chain of antibody M3-2
SEQ ID NO:3:Nucleotide sequence of variable region of light chain of antibody M3-2
SEQ ID NO:4: Amino acid sequence of variable region of light chain of antibody M3-2
SEQ ID NO:5:Nucleotide sequence of variable region of heavy chain of antibody M3-4
SEQ ID NO:6: Amino acid sequence of variable region of heavy chain of antibody M3-4
SEQ ID NO:7:Nucleotide sequence of variable region of light chain of antibody M3-4
SEQ ID NO:8: Amino acid sequence of variable region of light chain of antibody M3-4
SEQ ID NO:9:Nucleotide sequence of variable region of heavy chain of antibody M3-5
SEQ ID NO:10:Amino acid sequence of variable region of heavy chain of antibody M3-5
SEQ ID NO:11:Nucleotide sequence of variable region of light chain of antibody M3-5
SEQ ID NO:12:Amino acid sequence of variable region of light chain of antibody M3-5
SEQ ID NO:13:Nucleotide sequence of variable region of heavy chain of antibody M3-6
SEQ ID NO:14:Amino acid sequence of variable region of heavy chain of antibody M3-6
SEQ ID NO:15:Nucleotide sequence of variable region of light chain of antibody M3-6
SEQ ID NO:16:Amino acid sequence of variable region of light chain of antibody M3-6
SEQ ID NO:17:Nucleotide sequence of variable region of heavy chain of antibody M3-7
SEQ ID NO:18:Amino acid sequence of variable region of heavy chain of antibody M3-7
SEQ ID NO:19:Nucleotide sequence of variable region of light chain of antibody M3-7
SEQ ID NO:20:Amino acid sequence of variable region of light chain of antibody M3-7
SEQ ID NO:21:Nucleotide sequence of variable region of heavy chain of antibody M3-8
SEQ ID NO:22:Amino acid sequence of variable region of heavy chain of antibody M3-8
SEQ ID NO:23:Nucleotide sequence of variable region of light chain of antibody M3-8
SEQ ID NO:24:Amino acid sequence of variable region of light chain of antibody M3-8
SEQ ID NO:25:Nucleotide sequence of variable region of heavy chain of antibody M3-9
SEQ ID NO:26:Amino acid sequence of variable region of heavy chain of antibody M3-9
SEQ ID NO:27:Nucleotide sequence of variable region of light chain of antibody M3-9
SEQ ID NO:28:Amino acid sequence of variable region of light chain of antibody M3-9
SEQ ID NO:29:Nucleotide sequence of variable region of heavy chain of antibody M3-10
SEQ ID NO:30:Amino acid sequence of variable region of heavy chain of antibody M3-10
SEQ ID NO:31:Nucleotide sequence of variable region of light chain of antibody M3-10
SEQ ID NO:32:Amino acid sequence of variable region of light chain of antibody M3-10
SEQ ID NO:33:Nucleotide sequence of variable region of heavy chain of antibody M3-11
SEQ ID NO:34:Amino acid sequence of variable region of heavy chain of antibody M3-11
SEQ ID NO:35:Nucleotide sequence of variable region of light chain of antibody M3-11
SEQ ID NO:36:Amino acid sequence of variable region of light chain of antibody M3-11
SEQ ID NO:37:Nucleotide sequence of variable region of heavy chain of antibody M3-12
SEQ ID NO:38:Amino acid sequence of variable region of heavy chain of antibody M3-12
SEQ ID NO:39:Nucleotide sequence of variable region of light chain of antibody M3-12
SEQ ID NO:40:Amino acid sequence of variable region of light chain of antibody M3-12
SEQ ID NO:41:Nucleotide sequence of variable region of heavy chain of antibody M3-13
SEQ ID NO:42:Amino acid sequence of variable region of heavy chain of antibody M3-13
SEQ ID NO:43:Nucleotide sequence of variable region of light chain of antibody M3-13
SEQ ID NO:44:Amino acid sequence of variable region of light chain of antibody M3-13
SEQ ID NO:45:Nucleotide sequence of variable region of heavy chain of antibody M3-14
SEQ ID NO:46:Amino acid sequence of variable region of heavy chain of antibody M3-14
SEQ ID NO:47:Nucleotide sequence of variable region of light chain of antibody M3-14
SEQ ID NO:48:Amino acid sequence of variable region of light chain of antibody M3-14

Further, an antibody in a form normally existing in vivo can be prepared from scFv. For example, only the variable regions of the H chain and the L chain are amplified by PCR from a scFv plasmid. Each fragment is, for example, recombined into a plasmid having the H chain gene and/or the L chain gene of a human antibody, which thereby enables the formation of an antibody having a variable region on the scFv in a form normally existing in vivo. Specifically, for example, appropriate restriction enzyme cleavage sites are introduced at both ends of the gene fragment obtained when amplifying the variable regions of the H chain and the L chain from the plasmid, and they are combined with an appropriate restriction enzyme cleavage site on the plasmid having the H chain and/or the L chain of the human antibody, thereby replacing genes in the variable region without causing frame-shift. Thus, an antibody in a form normally existing in vivo which has a sequence of a variable region on a plasmid as it is, can be prepared. Further, a peptide containing at least one antigen-binding site formed of the variable region of the H chain or L chain of the antibody, or a combination thereof, Fab composed of a set of an H chain fragment and an L chain fragment, and (Fab′2) composed of two sets of H chain fragments and L chain fragments can also be prepared from the antibody.

The expression of the antibody of the present invention can be carried out by employing E. coli, yeast, insect cells, animal cells, and the like. For example, when an antibody is expressed in COS cell or CHO cell, pCDNA3.1(+) or pMAMneo (CLONETECH) can be used. For example, a gene of the H chain of the antibody obtained in the above method is incorporated into a multicloning site of pCDNA3.1(+), and a gene of the L chain is incorporated into pMAMneo. Then, an expression unit having a gene of the L chain between a promoter and poly A is incorporated into an adequate site of the vector having the H chain incorporated therein. Introduction of this vector into a COS cell, a CHO-K1, or a CHO DG44 by a conventional genetic engineering technique enables the production of the antibody of interest. Further, the expression unit of the DHFR gene is cleaved out from for example, pSV2/DHFR (Nature, 1981. Vol. 294, Lee F. et al.) into the above prepared vector, and is incorporated into a vector which expresses the H chain and the L chain. This vector is introduced into the CHO DG44 by a conventional genetic engineering technique. Thus selected cells can be used to significantly improve the productivity of antibodies by utilizing the DHFR gene amplification system using MTX.

Animal cells such as COS cell or CHO cell can be generally cultured in Dulbecco's Modified Eagle's Medium (DMEM) supplemented with 10% fetal bovine serum (FBS) under 5% CO₂ at 37° C. A method for introducing a gene into the COS cell may be electroporation, as well as a DEAE-dextran method and a method using a transfection reagent such as lipofectin.

At the time of production of the antibody of the present invention, the cells are preferably cultured in a serum-free medium in order to prevent the contamination of a serum-derived bovine antibody. COS and CHO cells which are not acclimatized in a serum-free medium but are cultured in serum media, are preferably cultured in serum-free DMEM. The antibody of the present invention, which is thus obtained in the culture supernatant, can be easily purified by a conventional method for purifying IgG antibodies using, for example, Protein A column and Protein G column.

The present invention will be more specifically described in the following examples. However, these Examples are not intended to limit the scope of the present invention. Abbreviations used in this application are as follows: Fv, antibody variable region; scFv, single-chain antibody variable region; Ab, antibody; mAb, monoclonal antibody; BSA, bovine serum albumin; IGF, insulin-like growth factor; Man1, mannose; Man2, mannobiose (Manα1-6Man); Man3, mannotriose [Manα1-6(Manα1-3)Man]; Man5, mannopentaose [Manα1-6(Manα1-3)Manα1-6(Manα1-3)Man]; GN2Man3, GlcNAcβ1-2Manα1-6(GlcNAcβ1-2Manα1-3)Man; GN2, di-N-acetylchitobiose (GlcNAcβ1-4GlcNAc); FG, Fucα1-2Gal; LNT, lacto-N-tetraose (Galβ1-3 GlcNAcβ1-3Galβ1-4Glc); and HRP, horseradish peroxidase.

### Examples

### (A) Materials and Methods

### (1) Materials

Escherichia coli strains used were the suppressor mutant strain TG1 and the nonsuppressor mutant strain TOP10F′ from Invitrogen (Carlsbad, Calif.). Helper phages M13KO7 were obtained from Amersham Biotech (UK). Bovine serum albumin (BSA), fetuin, asialo-fetuin, RNase A, RNase B, DPPE, and dipalmitoylphosphatidylcholine (DPPC) were purchased from Sigma-Aldrich (St. Louis, Mo.). Mannose (Man1), mannobiose (Man2; Manα1-6Man), mannotriose (Man3; Manα1-6(Manα1-3)Man), mannopentaose (Man5; Manα1-6(Manα1-3)Manα1-6(Manα1-3)Man), di-N-acetylchitobiose (GN2; GlcNAcβ1-4GlcNAc), lacto-N-tetraose (LNT; Galβ1-3GlcNAcβ1-3Galβ1-4Glc), Fucα1-2Gal(FG), GlcNAcβ1-2Manα1-6(GlcNAcβ1-2Manα1-3)Man(GN2Man3), Man1-BSA, and Man3-BSA were obtained from Dextra Laboratories (Reading, UK). Glucose-sp-biotin, Man3-sp-biotin, and Lewis b-sp-Biotin were obtained from GlycoTech (Gaithersburg, Md.). ABTS/H₂O₂ was obtained from Roche Diagnostics (Mannheim, Germany). Oxalic acid was obtained from Wako Pure Chemical (Osaka, Japan). Anti-M13 antibodies were purchased obtained from Amersham Bio-Science (Piscataway, N.J.) and Exalpha Biologicals (Watertown, Mass.). Anti-insulin-like growth factor (IGF)-I receptor scFv phage antibody was prepared from 1H7 mAb-derived scFv (Li, S.-L., Liang, S.-J., Guo, N., Wu, A. M., Fujita-Yamaguchi, Y. (2000) Cancer Immunol. Immunother. 49, 243-252) using Expression Module/Recombinant Phage Antibody System of Amersham Bio-Science.

### (2) Preparation of Artificial Glycolipids

Artificial glycolipids were synthesized from carbohydrates (Man1, Man2, Man3, Man5, GN2, LNT, FG, or GN2Man3) and DPPE by reductive amination according to previous methods (Stoll, M. S., Mizuochi, T., Childs, R. A., and Feizi, T. (1988) J. Biochem. 256, 661-665; Kenjo, A., Takahashi, M., Matsushita, M., Endo, Y., Nakata, M., Mizuochi, T., and Fujita, T. (2001) J. Biol. Chem. 276, 19959-19965; and Shimizu, Y., Nakata, M., Matsunuma, J., and Mizuochi, T. (2001) J. Chromatogr. B. Biomed. Sci. Appl. 754, 127-133). The reaction scheme is shown in FIG. 1A. Briefly, 940 μl of chloroform/methanol (1:1, v/v) solution containing 9.4 mg of DPPE and 200 μl of methanol solution containing 4 mg of sodium cyanoborohydride were added and mixed to 60 μl of aqueous solution containing each carbohydrate (1-2 mg). The reaction was carried out at 80° C. for 5 hours with occasional sonication. The artificial glycolipids in the reaction mixtures were then purified by HPLC on a silica gel column Shim-pack PREP-SIL (Shimadzu, Kyoto, Japan) and reversed-phase chromatography on a Bond Elut C18 cartridge column (Varian, Harbor City, Calif.) according to a previous method (Shimizu, Y., Nakata, M., Matsunuma, J., and Mizuochi, T. (2001) J. Chromatogr. B. Biomed. Sci. Appl. 754, 127-133). The purity and structure of each artificial glycolipid were confirmed with TLC and MALDI-TOF mass spectrometry.

### (3) Construction of Phage Library

cDNA, PCR Amplification, and Cloning of VH and VL Gene Repertoire To Construct Primary Phage Libraries

PCR templates used in this study were human spleen cDNA purchased from BioChain (Hayward, Calif.) and human leukocyte cDNA purchased from BD Biosciences (Palo Alto, Calif.). The human immunoglobulin variable regions were amplified by PCR using an equimolar mixture of appropriate forward and reverse primers having sequences in common with this gene family, according to previously published information (Sbalattero, D. & Bradbury, A. (1998) Immunotechnology 3, 271-278).

5′-primer:
(SEQ ID NO: 49)
VHfl
AGCGCGGCCtctagaCAGGTGCAGCTGCAGGAGTCSG
(SEQ ID NO: 50)
VHf2
AGCGCGGCCtctagaCAGGTACAGCTGCAGCAGTCA
(SEQ ID NO: 51)
VHf3
AGCGCGGCCtctagaCAGGTGCAGCTACAGCAGTGGG
(SEQ ID NO: 52)
VHf4
AGCGCGGCCtctagaGAGGTGCAGCTGKTGGAGWCY
(SEQ ID NO: 53)
VHf5
AGCGCGGCCtctagaCAGGTCCAGCTKGTRGAGTCTGG
(SEQ ID NO: 54)
VHf6
AGCGCGGCCtctagaCAGRTCACCTTGAAGGAGTCTG
(SEQ ID NO: 55)
VHf7
AGCGCGGCCtctagaCAGGTGCAGCTGGTGSARTCTGG
(SEQ ID NO: 56)
Vkf1
GACATCCRGDTGACCCAGTCTCC
(SEQ ID NO: 57)
Vkf2
GAAATTGTRWTGACRCAGTCTCC
(SEQ ID NO: 58)
Vkf3
GATATTGTGMTGACBCAGWCTCC
(SEQ ID NO: 59)
Vkf4
GAAACGACACTCACGCAGTCTC
(SEQ ID NO: 60)
Vlf1
CAGTCTGTSBTGACGCAGCCGCC
(SEQ ID NO: 61)
Vlf2
TCCTATGWGCTGACWCAGCCAC
(SEQ ID NO: 62)
Vlf3
TCCTATGAGCTGAYRCAGCYACC
(SEQ ID NO: 63)
Vlf4
CAGCCTGTGCTGACTCARYC
(SEQ ID NO: 64)
Vlf5
CAGDCTGTGGTGACYCAGGAGCC
(SEQ ID NO: 65)
Vlf6
CAGCCWGKGCTGACTCAGCCMCC
(SEQ ID NO: 66)
Vlf7
TCCTCTGAGCTGASTCAGGASCC
(SEQ ID NO: 67)
Vlf8
CAGTCTGYYCTGAYTCAGCCT
(SEQ ID NO: 68)
Vlf9
AATTTTATGCTGACTCAGCCCC
3′-primer:
(SEQ ID NO: 69)
VHr1
CATTGAGGAGACRGTGACCAGGGTG
(SEQ ID NO: 70)
VHr2
CATTGAGGAGACGGTGACCAGGGTT
(SEQ ID NO: 71)
VHr3
CATTGAAGAGACGGTGACCATTGT
(SEQ ID NO: 72)
VHr4
CATTGAGGAGACGGTGACCGTGGTCC
(SEQ ID NO: 73)
VHr5
CATGGTTGGGGCGGATGCACTCC
(SEQ ID NO: 74)
VHr6
CATSGATGGGCCCTTGGTGGARGC
(SEQ ID NO: 75)
VKr1
TTTGATTTCCACCTTGGTCC
(SEQ ID NO: 76)
VKr2
TTTGATCTCCASCTTGGTCC
(SEQ ID NO: 77)
VKr3
TTGATATCCACTTTGGTCC
(SEQ ID NO: 78)
VKr4
TTTAATCTCCAGTCGTGTCC
(SEQ ID NO: 79)
Vlr1
TAGGACGGTSASCTTGGTCC
(SEQ ID NO: 80)
V1r2
GAGGACGGTCAGCTGGGTGC
Primer for CDR shuffling
(SEQ ID NO: 81)
VH(FWR)fl
GGACACGGCCGTRTATTACTGT
(SEQ ID NO: 82)
VH(FWR)f2
GGACAYGGCCATGTATTACTGT
(SEQ ID NO: 83)
Vk(FWR)f1
ATCAGYWGNSTGSARSCTGARGAT
(SEQ ID NO: 84)
Vk(FWR)f2
ATCAATWGNSTGSARSCTGARGAT
(SEQ ID NO: 85)
Vl(FWR)f1
GAYGAGGCTGACTATTACTGT
(SEQ ID NO: 86)
Vl(FWR)f2
GATGAATCTGATTATTACTGT
(SEQ ID NO: 87)
Vl(FWR)f3
GATGAGGCTGAGTATCATTGT
(SEQ ID NO: 88)
Vl(FWR)f4
GATGAGAGTGACTACCACTGT
(SEQ ID NO: 89)
VH(FWR)r1
ACAGTAATAYACGGCCGTGTCC
(SEQ ID NO: 90)
VH(FWR)r2
ACAGTAATACATGGCCRTGTCC
(SEQ ID NO: 91)
Vk(FWR)r1
ATCYTCAGSYTSCASNCWRCTGAT
(SEQ ID NO: 92)
Vk(FWR)r2
ATCYTCAGSYTSCASNCWATTGAT
(SEQ ID NO: 93)
Vl(FWR)r1
ACAGTAATAGTCAGCCTCRTC
(SEQ ID NO: 94)
Vl(FWR)r2
ACAGTAATAATCAGATTCATC
(SEQ ID NO: 95)
Vl(FWR)r3
ACAATGATACTCAGCCTCATC
(SEQ ID NO: 96)
Vl(FWR)r4
ACAGTGGTAGTCACTCTCATC

Reaction mixtures (50 μl) contained 0.5 μl of the cDNA solution, 25 pmol of each of forward primer and reverse primer, 200 μM dNTPs, 10 mM KCl, 10 mM (NH₄)₂SO₄, 20 mM Tris-HCl (pH 8.8), 2 mM MgCl₂, 100 μg/ml BSA, and 1 unit of KOD plus DNA polymerase (Toyobo, Osaka, Japan). The PCR condition was optimized for each primer set. The first round PCR products purified by agarose gel electrophoresis were mixed in the same quantity as used for templates in the following PCR to amplify immunoglobulin variable regions. The resulting DNA fragments of the variable regions were purified and digested with appropriate restriction enzymes followed by insertion into pVH-Hyg or pVL-Amp vectors. After ligation, the resulting phagemid DNA was electroporated into E. coli DH10B strain harboring F′ plasmid derived from E. coli XL1-Blue, and colonies were grown at 30° C. on agar plates containing 1% glucose and hygromycin (50 μg/ml) or ampicillin (50 μg/ml). The colonies were collected and converted to phages bearing VH or VL genes by superinfection with helper phage M13KO7 in 2× YT medium containing 1% glucose at 30° C. overnight. The phages were collected by centrifugation and precipitation with polyethylene glycol (PEG) according to a standard protocol.

Recombination and Generation of Secondary Phage scFv Library

The F′ plasmid derived from E. coli XL1-Blue strain was introduced into E. coli NS3529 strain, which expresses Cre-recombinase constitutively, to obtain E. coli NS3529/F′ strain. The NS3529/F′ strain was infected with VH and VL phages at MOI=50, and was left still at 37° C. for 1 hour without shaking, and was then superinfected with M13KO7 helper phages at MOI=20. After samples were incubated at 37° C. for 1 hour, ampicillin and kanamycin were added at 50 μg/ml and 25 μg/ml, respectively. During overnight culture at 37° C., Cre/lox recombination resulted in formation of scFv genes because of insertion of a VH fragment into the pVL-Amp vector in the NS3529/F′ cells. Growing XL1-Blue strain was infected with the recombinant phages obtained from the culture at MOI<0.1, and was left still at 37° C. for 1 hour. ScFv displaying phages fused with a coat protein g3p were obtained from the strain which was cultured in 2× YT medium (glucose free) containing 20 μg/ml chloramphenicol, 50 μg/ml ampicillin and 25 μg/ml kanamycin at 25° C. overnight. The phages which were concentrated by PEG precipitation were suspended in SM buffer containing 1% gelatin and 6% DMSO, and were stored at −80° C.

### (4) Phage Selection Methods

### Panning Procedures

The library was subjected to four rounds of panning. Fifty and sixteen wells of a 96-well plate were coated with Man3-DPPE (2 μg/well) and used for first and second panning, respectively. Five and two wells coated with Man3-DPPE (2 μg/well) were used for third and fourth panning, respectively. Phage selection was basically carried out according to previously published procedures (Marks, J. D., Hoogenboom, H. R., Bonnet, T. P., McCafferty, J., Griffiths, A. D., and Winter, G. (1991) J. Mol. Biol. 222, 581-597) with some modifications. Coating of wells with Man3-DPPE is described briefly. Wells are applied with 50 μl of Man3-DPPE (40 μg/ml methanol solution) and the solvent is dried at 37° C., followed by incubation with 150 μl of Tris-HCl buffer (pH 7.4) containing 0.15 M NaCl and 3% BSA (blocking buffer) at 4° C. overnight. The wells were rinsed twice with 50 μl of 0.2% Tween 20/TBS (TBS-T), and once with 200 μl of TBS. 50 μl of phage suspension in TBS containing 0.1% Tween 20 and 1.4% BSA was added to the wells, which were then incubated at 37° C. for 60 minutes under shaking. After the wells were washed 3 times with 200 μl of TBS-T and twice with 200 μl of TBS, bound phages were eluted by addition of 50 μl of 100 mM triethylamine and incubation at 25° C. for 10 minutes. Then, the resultant product was neutralized by mixing with 100 μl of neutralizing solution (1 M Tris-HCl (pH 7.4):3% BSA/TBS=2:1, v/v) in wells of the control plate, which had been treated with the blocking buffer. Bound phages were further eluted by addition of 50 μl of 100 mM triethylamine and incubation at 25° C. for 20 minutes and were recovered in the neutralization solution as described above. Eluted phages were used to infect 100 μl of logarithmic growing E. coli TG1 strain at 37° C. for 1 hour. Infected bacteria were grown on LB agar plates (medium) (diameter=10 cm) containing 1 mM NaOH, 0.1% glucose, and carbenicillin (50 μg/ml) at 25° C. for 16 hours, and then were collected from the plates using a spreader after addition of 2 ml of LB-10 mM Tris-HCl (pH 7.5) (SBS) per plate. 1 ml out of the 38 ml of this suspension was inoculated into 40 ml of SBS containing carbenicillin and grown with shaking at 37° C. for 2 hours. 40 μl of solution containing 3.5×10⁹ pfu. of helper phages was added and the resultant was incubated at 37° C. for 1 hour without shaking. Then, kanamycin (25 μg/ml)/chloramphenicol (10 μg/ml) was added, and the resultant was incubated with rotation at 25° C. for 40 hours. Thus, phages were collected. Phage particles were concentrated by PEG-precipitation, and were dissolved in 400 μl of TBS, 400 μl of 3% BSA/TBS, and 40 ml of 10% Tween 20/TBS by incubation at 37° C. for 1 hour. After centrifugation (at 18,000 g at 4° C. for 5 minutes), 800 μl of phage suspension were recovered and used for second panning. Second, third, and fourth panning were carried out similarly to first panning except for washing conditions. Bacteria picked from single colonies after the fourth panning were grown in 50 μl of SBS/carbenicillin in 96 well plates at 37° C. for 1 hour with rotation, to which 50 μl of helper phages were added and incubated at 37° C. for 1 hour with rotation. After SBS/kanamycin/chloramphenicol mixture was added at 50 μl/well, phage suspensions were collected through incubation at 25° C. for 16 hours with rotation and centrifugation (at 200 g at 4° C. for 15 minutes). 50 μl of the supernatants were added to wells containing 100 μl of 3% BSA/TBS, incubated at 37° C. for 1 hour, and kept at 4° C.

### Screening of Phage Clones Expressing Antibodies Directed Against Man3-DPPE (By ELISA)

Analysis of binding of phages to Man3-DPPE was performed by ELISA using bacterial supernatants containing phages. A 96-well plate was coated with Man3-DPPE (1 μg/well) as described above and blocked by incubation with 150 μl of 3% BSA/TBS at 4° C. overnight. Control plates were prepared as above without antigen. 75 μl of phage suspensions were added to the wells and incubated at 37° C. for 1 hour. The wells were washed 5 times with 200 μl of TBS-T. Bound phage antibodies were detected by incubation with horseradish peroxidase (HRP)-labeled anti-M13 antibody at 37° C. for 1 hour, after which they were washed 10 times with 200 μl of TBS-T and once with 200 μl of TBS. Peroxidase activity was detected by reaction with ABTS/H₂O₂ for 30 minutes and termination with 1% oxalic acid. Then, the absorbance was measured with a BIO-RAD plate-reader at 415 nm.

### (5) Characterization of Phage Antibodies

### Colony PCR and Determination of DNA Sequences

scFv genes were amplified from respective E. coli TG1 colonies infected with phages by PCR with a primer set (forward primer Cm-f: 5′-TGTGATGGCTTCCATGTCGGCAGAATGCT-3′ (SEQ ID NO: 97), and reverse primer g3-r: 5′-GCTAAACAACTTTCAACAGTCTATGCGGCAC-3′ (SEQ ID NO: 98)). After preheating the sample at 94° C. for 2 minutes, PCR was carried out with 35 cycles under conditions of denaturing at 94° C. for 20 seconds, annealing at 53° C. for 20 seconds, and extension reaction at 68° C. for 1 minute. After purification and confirmation of the length of the PCR product in 2% agarose gel electrophoresis, the resulting scFv genes were subjected to DNA sequencing. DNA sequences of scFvs were determined using a 3730 DNA analyzer (Applied Biosystem, Foster City, Calif.).

### Binding Assays on TLC Plates

400 pmol of artificial glycolipids dissolved in chloroform/methanol/water (50:55:18, v/v) were dot-blotted onto TLC plates. Alternatively, mixtures containing 1 μg each of artificial glycolipids were spotted on aluminum-backed silica gel 60 high-performance TLC plates (10 cm-length; Merck, Darmstadt, Germany), and were developed with a solvent system of chloroform/methanol/water (60:35:8, v/v). After drying, these plates were soaked for 30 seconds in n-hexane containing 0.1% (w/v) Plexigum P28 (Sigma-Aldrich) and then blocked with 3% BSA/TBS at room temperature for 1 hour followed by washing with TBS. The plates were then overlaid with phage antibody (10¹³ cfu/ml) at 4° C. overnight followed by overlay with mouse anti-M13 phage coat protein (p8) IgG (5000-fold dilution) at room temperature for 1 hour. The binding of phage antibodies to artificial glycolipids was detected by a combination of HRP-labeled anti-mouse IgG (MBL, Nagoya, Japan) and a chemiluminescent reagent (ECL™ Western blotting detection reagents, Amersham Bio-Sciences, Buckinghamshire, UK) according to the manufacturer's instructions.

### Determination of Carbohydrate Specificity of Phage Antibodies Through Examination of Their Reactivity to Natural Glycoproteins

Fetuin, asialo-fetuin, RNase A, and RNase B (5 μg/lane) were separated by SDS-polyacrylamide gel electrophoresis (10% gel) and blotted onto a PVDF membrane. The SDS-PAGE gel was stained with Coomassie Brilliant Blue. The reactivity of phage antibody to natural glycoproteins was examined by Western blotting with M3-7 phage antibody followed by HRP-labeled anti-M13 phage antibody as described above.

Expression and Partial Purification of scFv Proteins

Each isolated phage clone was subjected to infection to E. coli TOP10F′ strain containing chaperon/repressor vector. The TOP10F′ cells infected with M3-7 or a control anti-FLAG phage were cultured in 200 ml of 2× YT medium containing 50 μg/ml ampicillin, 50 μg/ml spectinomycin and 1 mM isopropyl-thio β-D-galactopyranoside (IPTG) at 30° C. for 3 hours. Supernatants, periplasmic fractions, or whole cell extracts of the TOP10F′ cells were collected by differential centrifugation. Whole cell extracts, collected from 40 ml of the culture solution, were poured into 1 ml of 50 mM Tris-HCl (pH 8) containing 6 M guanidine-HCl, 5% glycerol, 0.5 mM PMSF, and 0.1 mM DTT, and kept on ice for 1 hour for solubilization. After centrifugation (at 18,800 g for 30 minutes), the supernatants were dialyzed against 10 mM HEPES (pH 7.4) containing 150 mM NaCl. Aggregates were removed by centrifugation and then the supernatants were subjected to SDS-PAGE/Western blotting analysis. A total protein amount of 18 μg/lane was separated by SDS-PAGE (4-20% acrylamide gel) (Daiichi Pure Chemicals, Tokyo, Japan) under reducing conditions and then transferred to a PVDF membrane. After blocking with 3% BSA/PBS, scFv proteins expressed were detected with an HRP-labeled anti-E-Tag antibody (Amersham Bio-Sciences, Japan). The scFv protein samples were used for SPR analysis.

Binding of scFv Proteins to Synthetic Glycoproteins (By ELISA)

scFv proteins were produced by the following method for use in the specificity assay to various BSA-conjugated oligosaccharides. TOP10F′-FS2 cells were infected with Man3-specific phages, and cultured in 5 ml of SBS with 1% glucose, 50 μg/ml carbenicillin, and 50 μg/ml spectinomycin at 25° C. for 16 hours. Next, the infected cells were collected by centrifugation, then suspended, and cultured in 40 ml of SBS with carbenicillin and spectinomycin at 30° C. for 3 hours. After 1 mM IPTG was added, the suspension was cultured at 30° C. for 16 hours to induce scFv proteins, and the supernatant was collected. After dot-blotting onto a nitrocellulose membrane, HRP-labeled anti-E-tag antibody was added to confirm the expression of scFv proteins.

The ELISA plate was coated with 50 μl of BSA-conjugated oligosaccharide (10 μl/ml) at 4° C. for 16 hours. The supernatant of the culture solution was added to the wells (50 μl/well) and left at 37° C. for 2 hour. Binding of scFv proteins to oligosaccharides was assayed according to the above method with use of HRP-labeled anti-E-tag antibody.

Characterization of Phage Antibodies and scFv Proteins (Surface Plasmon Resonance, SPR)

SPR analyses were performed at 25° C. Solutions were freshly prepared, degassed, and filtered through 0.22-μm pore filter. Binding property of phage antibodies were determined by SPR using BIAcore X (Pharmacia Biosensor). Used in this analysis were HPA sensor chips where aliphatic chains were covalently bound to a gold surface, and 10 mM HEPES (pH 7.4) containing 150 mM NaCl (HBS) as a running buffer. Immobilization of artificial glycolipids on the HPA sensor chip surface was carried out according to the manufacturer's protocol. Briefly, a lipid monolayer with carbohydrates on the surface was formed on the HPA sensor chip by adding artificial glycolipids bearing liposomes. Before immobilization, the surface was cleaned by injection of nonionic detergent, 40 mM MEGA9 (Dojindo, Kumamoto, Japan) and 50% ethanol at a flow rate of 5 μl/min. DPPC or Man3-DPPE/DPPC (1:10, mol/mol) was then applied to the sensor chip surface at a low flow rate of 2 μl/min. To remove multilamellar structures from the lipid surface, 5 μl of 10 mM NaOH was injected at a flow rate of 5 μl/min. Phage antibody samples that had been dialyzed against HBS were allowed to flow for 10 minutes over the surface of the chips at a flow rate of 5 μl/min. To examine the competitive inhibitory effect on SPR, phage antibody samples were mixed with 10 mM or 100 mM D-mannose or α-methyl-D-mannopyranoside (Nacalai Tesque, Kyoto, Japan), and then incubated at 4° C. for 1 hour.

SPR analyses of scFv protein samples were performed at a flow rate of 5 μl/min with BIAcore 3000 using the Sensor chip HPA or the Sensor chip SA (Streptavidin). Immobilization of DPPC, Man3-DPPE/DPPC (1:10, mol/mol), Man2-DPPE/DPPC (1:10, mol/mol), Man5-DPPE/DPPC (1:10, mol/mol) to HPA sensor chips was performed as described above. The amounts of immobilized ligands were calculated from the molar ratio of the DPPE conjugates included in the lipid layer, which indicated that DPPC, Man2-DPPE, Man3-DPPE, and Man5-DPPE were 0.43 pmol, 0.31 pmol, 0.48 pmol, and 0.30 pmol, respectively. HBS containing 0.01% Tween 20 (HBS-T) was used as a running buffer when SA sensor chips were used. SA sensor chip surface was activated with three consecutive 1-min injections of activating buffer (1 M NaCl containing 50 mM NaOH) prior to immobilization of biotinylated ligands. Glucose-sp-Biotin, Man3-sp-Biotin, and Lewis b-sp-Biotin (0.1 mM) were diluted with HBS-T to 0.05 μM, 10 μl of which were injected manually at a flow rate of 1 μl/min until RU of 100-150 is achieved. Unlike artificial glycolipids used above, these ligands contained a spacer (sp) consisting of a structure of —O(CH₂)₃NHCO(CH₂)₅NH—. The amounts of immobilized biotinylated carbohydrates were estimated to be 0.25 pmol, 0.23 pmol, and 0.13 pmol, respectively. The scFv samples that had been dialyzed against HBS-T were injected at a flow rate of 1 μl/min for 4 minutes over the surface of the chips. After one sample was assayed, the sensor chip surfaces were regenerated by treating with 10 mM glycine-HCl (pH 1.5) for 1 minute, followed by washing with a continuous flow of HBS-T before the next sample was injected.

### (B) Results

(1) Screening of scFv-Displaying Phages Directed against Man3-DPPE

The phage displayed human scFv library was subjected to four rounds of panning against Man3-DPPE. The structure of Man3-DPPE is presented in FIG. 1B. Man3-DPPE (2 μg/well) was used for panning according to abovementioned methods. Of 672 clones screened by ELISA using Man3-DPPE (1 μg/well) as an antigen, 40 or more clones was ELISA-positive with S/N of >2. Of those, 25 positive clones encoding scFvs were selected as candidates for phage antibodies directed against the Man3 structure. DNA sequencing of scFv regions of those phage clones revealed that all the clones analyzed had different sequences. Characteristics of 12 clones are shown in Table 1.

TABLE 1
Man3-Specific Single Chain Antibodies

| | Sequences of CDR3 | V_{H} | V_{L} |
|---|---|---|---|
| Clones | regions of VH Chains | family | family |
| M3-2 | DISKAHDY | V_{H}II | VκIII |
| M3-4 | GGGKEVTVLGLPVGWDYYGMDI | V_{H}IV | VκI |
| M3-5 | ORWSNFDY | V_{H}II | VκIII |
| M3-6 | SGLYGTTPSTFDH | V_{H}IV | VλI |
| M3-7 | ADPGYMIFNWFDP | V_{H}IV | VλI |
| M3-8 | AIKADSGFYLLDV | V_{H}I | VλI |
| M3-9 | DVVLGSGSTDQ | V_{H}III | VκIV |
| M3-10 | QFQEHTISANEQPEDGHGSVL | V_{H}III | VκIV |
| M3-11 | DYNPRSFDH | V_{H}II | VλI |
| M3-12 | NAPPYTHYFDQ | V_{H}III | VκIV |
| M3-13 | DTGTYARMSGMDV | V_{H}III | VκI |
| M3-14 | DAGNRGSYDWFDP | V_{H}IV | VκI |

### (2) Carbohydrate Specificity of Phage Clones

In order to quickly identify which phage antibodies have specificity and high affinity for the antigen used, it is necessary to establish methods suitable for characterizing scFv-presenting phages as phage antibodies. Although expression, isolation, and characterization of scFv proteins are ultimately required, expression and isolation of scFv proteins are labor-intensive and time-consuming, and yet primary information on phage clones is essential to select candidate clones for further characterization. For this purpose, a set of artificial glycolipids was individually spotted on TLC plates (FIG. 2A), and then phage antibodies were overlaid on the plates to allow their binding to the artificial glycolipids. These experiments revealed that phage antibodies showed specificity to Man3, as well as to Man2 and Man5 to a lesser extent as compared to Man3 (FIG. 2A). In addition, since binding to GN2Man3 and DPPE was not observed, it was also demonstrated that the phage antibodies examined bound to non-reducing mannose residues but not to the lipid portion of artificial glycolipids. The structures of artificial glycolipids used in these experiments are illustrated in FIG. 2B.

### (3) Binding of Phage Antibodies to Natural Glycoproteins

Phage antibodies which were isolated by using Man3-DPPE as a target antigen were shown to have specificity to non-reducing mannose residues of artificial glycolipids. Therefore, it was next examined whether these phage antibodies can bind to the non-reducing mannose residues of glycoproteins. It was clearly shown that M3-7 phage antibody bound to RNase B carrying high mannose type oligosaccharides but not to non-glycosylated RNase A or to fetuin carrying complex type and O-linked oligosaccharides (FIG. 3).

(4) SPR Analyses of Man3-Specific scFv-Displaying Phage Antibodies

After specificity of phage antibodies to Man3 was confirmed by ELISA and TLC-overlay assays, SPR was used to observe the binding kinetics of phage antibodies. A sensorgram showing the binding of M3-7 phage to Man3-DPPE (the sensorgram was obtained by subtracting the binding of phage antibody to Man3-DPPE by that of DPPC alone) confirmed the Man3-specific binding property of the examined phage antibody (FIG. 4; sensorgram A). This result was further supported by the fact that (i) the binding of phage antibody was competitively inhibited by mannose (sensorgram C) or α-methyl-D-mannoside, and (ii) a control phage antibody which displayed human IGF-I receptor specific scFv did not bind to Man3-DPPE (sensorgram B).

(5) Expression and Characterization of Man3-Specific scFvs as Soluble Proteins

scFv proteins were expressed as soluble proteins for further analysis with regard to their carbohydrate specificity. The content of scFv proteins was compared among supernatants, periplasmic fractions, and whole cell extracts by SDS-PAGE and subsequent immunoblotting with anti-E-tag antibody. As a result, it was revealed that whole cell extract fractions contained the most scFv proteins expressed. The whole cell fractions were thus used for ELISA and SPR analyses (FIG. 5). Although the fractions used were unpurified (FIG. 5 Left), they contained E-tag reactive scFv proteins (FIG. 5 Right). The amounts of scFv proteins expressed by E. coli cells that had been infected with phages under the same condition to that of this study can also be considered to be within a similar range (FIG. 5 Right). The binding abilities of scFv proteins M3-7 and M3-8 to mannose residues were confirmed by ELISA with use of synthetic glycoproteins (FIG. 6).

(6) SPR Analysis of Man3-Specific scFvs as Soluble Proteins

scFv protein samples (FIG. 5) were used to analyze the binding kinetics of Man3-specific scFv proteins by SPR using immobilized artificial glycolipids (FIG. 7). As a result, the scFv protein derived from the M3-7 phage clone showed similar relative kinetic of binding to Man3-DPPE and Man5-DPPE, which were higher than the binding kinetic to DPPE. On the other hand, the binding kinetic to Man2-DPPE was lower than the binding kinetic to DPPE (FIG. 7).

SPR Analyses of biotinylated carbohydrates were also carried out. In view of the binding kinetics of scFv protein samples to Man3-sp-biotin, Lewis b-sp-biotin, and glucose-sp-biotin, the scFv protein M3-7 bound to Man3 at much higher affinity than to Lewis b which is structurally irrelevant (the affinity of the scFv protein M3-7 to Lewis b was very low but significant) (FIG. 8). On the other hand, these scFv proteins did not appear to bind to glucose used as a control. The sensorgram of anti-FLAG scFv protein should be regarded as a control.

The sensorgrams of the bindings of scFv proteins to Man3-DPPE (FIG. 7) and Man3-biotin (FIG. 8) were respectively subtracted by the sensorgrams on DPPE (FIG. 9A) and glucose-biotin (FIG. 9B). From these two types of results, the specific binding to Man3 was compared (FIG. 9). These data show several facts regarding relative affinities between scFv proteins examined. For example, the scFv protein obtained from M3-7 has an affinity to Man3 structure.

### INDUSTRIAL APPLICABILITY

The present invention provides antibodies which recognize non-reducing mannose residues, and nucleic acids encoding these antibodies. The antibodies of the present invention are able to bind, with specificity and high affinity, to various carbohydrates including carbohydrate antigens, individual recognition of which is not feasible. Further, gene manipulation of single chain antibodies of the present invention enables production of modified single chain antibodies, and mass production of single chain antibodies with ease. In addition, gene manipulation of the antibodies obtained by the present invention enables carbohydrate-specific delivery of proteins such as toxins and enzymes.

### BRIEF DESCRIPTION OF THE DRAWINGS

FIG. 1 shows synthesis of artificial glycolipids (A) and Man3-DPPE (B). Artificial glycolipids were synthesized from oligosaccharides, such as Man1, Man2, Man3, Man5, GN2, LNT, FG, and GN2Man3 (whose structures are shown in FIG. 2C), and dipalmitoylphosphatidylethanolamine (DPPE) by reductive amination.

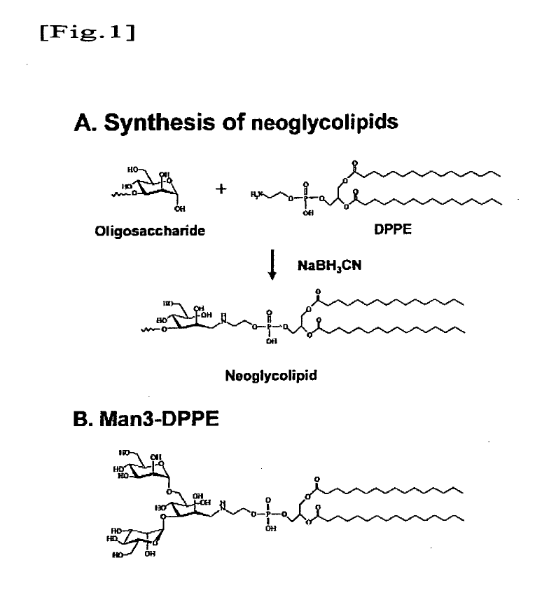

FIG. 2 shows carbohydrate specificity of phage antibodies screened by panning and ELISA with Man3-DPPE. (A) After various artificial glycolipids were dot-blotted on TLC plates, each plate was overlaid with anti-Man3 phage antibodies, M3-7, or M3-8, followed by detection of phages. (B) shows the artificial glycolipids used in this study. Man1, mannose; Man2, mannobiose (Manα1-6Man); Man3, mannotriose [Manα1-6(Manα1-3)Man]; Man5, mannopentaose [Manα1-6(Manα1-3)Manα1-6(Manα1-3)Man]; GN2Man3, GlcNAcβ1-2Manα1-6(GlcNAcβ1-2Manα1-3)Man; GN2, di-N-acetylchitobiose (GlcNAcβ1-4GlcNAc); FG; Fucα1-2Gal; LNT, lacto-N-tetraose (Galβ1-3GlcNAcβ1-3Galβ1-4Glc)

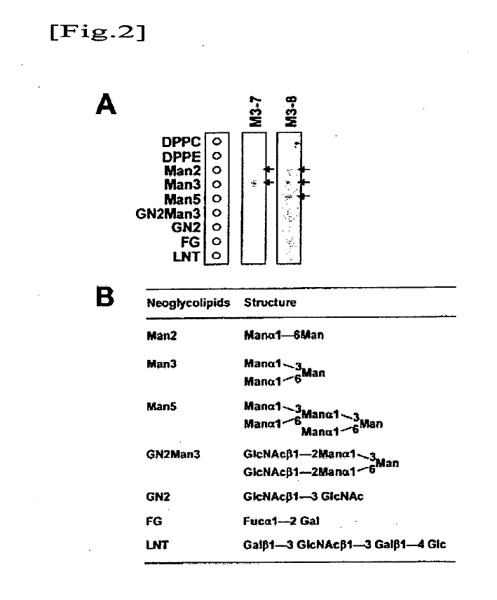

FIG. 3 shows binding of phage antibodies to natural glycoproteins. Reactivity of M3-7 phage antibody to natural glycoproteins (fetuin, asialofetuin, RNase A, and RNase B) was examined by Western blotting. The protein bands were stained with Coomassie Brilliant Blue. Western blotting with M3-7 phage antibody and HRP-labeled anti-M13 phage antibody was carried out to identify the band where the phage antibodies was bound.

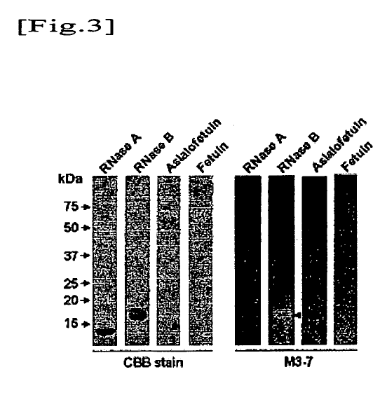

FIG. 4 shows SPR analysis of Man3-specific scFv-displaying phage antibodies. M3-7 phage antibody (A) was subjected to SPR analysis with Man3-DPPE. Anti-IGF-I receptor scFv-phage was used as a negative control (B). A competitive inhibition test was performed with M3-7 phage antibody in the presence of 100 mM mannose (C). The sensorgrams shown are specific binding to Man3, which were obtained by subtracting the sensorgrams on DPPC from those obtained on specific Man3-DPPE.

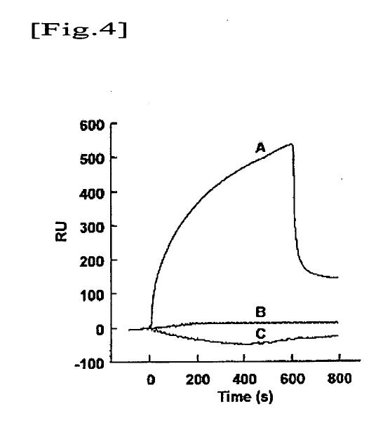

FIG. 5 shows expression of Man3-specific scFv as a soluble protein. Left: SDS-PAGE of scFv protein (M3-7) expressed in E. coli TOP10F′ as well as anti-FLAG scFv protein as a control was carried out. The gel was stained with Coomassie Brilliant Blue. Right: Western blotting of scFv proteins with an anti-E-tag antibody was performed.

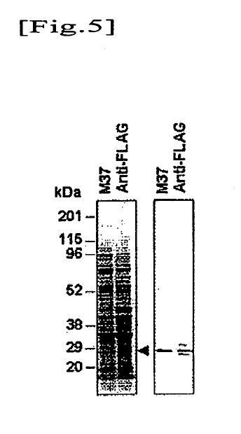

FIG. 6 shows binding of scFv proteins to Man3-BSA (examined by ELISA). scFv proteins (M3-7 and M3-8) were analyzed by ELISA. Reactivities of respective scFv proteins to BSA (a), Man1-BSA (b), Man3-BSA (c), and scFv protein-free BSA (d) were determined.

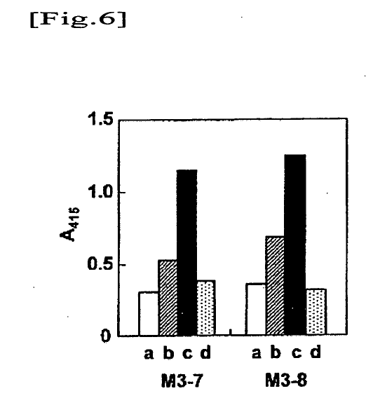

FIG. 7 shows SPR analysis of Man3-specific scFv proteins on chip immobilized with Man2-DPPE, Man3-DPPE, or Man5-DPPE.

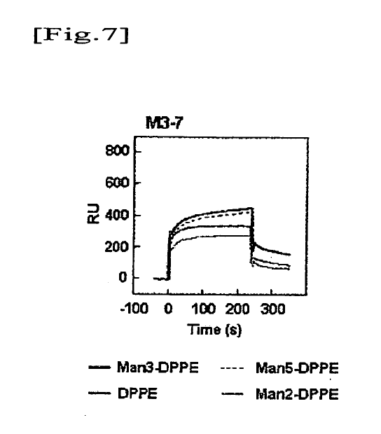

FIG. 8 shows SPR analysis of Man3-specific scFv proteins on chip immobilized with Man3-sp-biotin, Lewis b-sp-biotin, or glucose-sp-biotin. The thick line indicates Man3-sp-biotin; the dotted line indicates Lewis b-sp-biotin; and the thin line indicates glucose-sp-biotin.

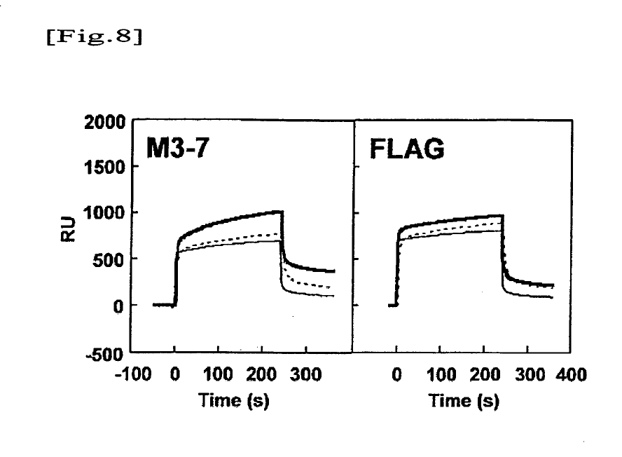

FIG. 9 shows comparison of the bindings of scFv proteins to Man3 moiety. The comparison was made between two types of sensorgrams of scFv proteins with respect to Man3-DPPE (FIG. 7) and Man3-biotin (FIG. 8). Specific bindings were obtained by subtracting the sensorgrams on DPPE (FIG. 9A) or glucose-biotin (FIG. 9B) from the respective sensorgrams.

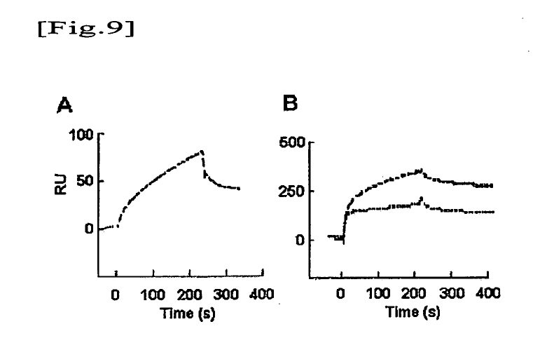

1. An antibody of any of the following (1) to (14):
(1) an antibody recognizing a non-reducing mannose residue, comprising
the amino acid sequence of SEQ ID NO: 2 or an amino acid sequence, wherein one or a few amino acids have been deleted, substituted, or added in the amino acid sequence of SEQ ID NO: 2, as an amino acid sequence of heavy chain variable region, and the amino acid sequence of SEQ ID NO: 4 or an amino acid sequence, wherein one or a few amino acids have been deleted, substituted, or added in the amino acid sequence of SEQ ID NO: 4, as an amino acid sequence of light chain variable region;
(2) an antibody recognizing a non-reducing mannose residue, comprising
the amino acid sequence of SEQ ID NO: 6 or an amino acid sequence, wherein one or a few amino acids have been deleted, substituted, or added in the amino acid sequence of SEQ ID NO: 6, as an amino acid sequence of heavy chain variable region, and the amino acid sequence of SEQ ID NO: 8 or an amino acid sequence, wherein one or a few amino acids have been deleted, substituted, or added in the amino acid sequence of SEQ ID NO: 8, as an amino acid sequence of light chain variable region;
(3) an antibody recognizing a non-reducing mannose residue, comprising
the amino acid sequence of SEQ ID NO: 10 or an amino acid sequence, wherein one or a few amino acids have been deleted, substituted, or added in the amino acid sequence of SEQ ID NO: 10, as an amino acid sequence of heavy chain variable region, and the amino acid sequence of SEQ ID NO: 12 or an amino acid sequence, wherein one or a few amino acids have been deleted, substituted, or added in the amino acid sequence of SEQ ID NO: 12, as an amino acid sequence of light chain variable region;
(4) an antibody recognizing a non-reducing mannose residue, comprising
the amino acid sequence of SEQ ID NO: 14 or an amino acid sequence, wherein one or a few amino acids have been deleted, substituted, or added in the amino acid sequence of SEQ ID NO: 14, as an amino acid sequence of heavy chain variable region, and the amino acid sequence of SEQ ID NO: 16 or an amino acid sequence, wherein one or a few amino acids have been deleted, substituted, or added in the amino acid sequence of SEQ ID NO: 16, as an amino acid sequence of light chain variable region;
(5) an antibody recognizing a non-reducing mannose residue, comprising
the amino acid sequence of SEQ ID NO: 18 or an amino acid sequence, wherein one or a few amino acids have been deleted, substituted, or added in the amino acid sequence of SEQ ID NO: 18, as an amino acid sequence of heavy chain variable region, and the amino acid sequence of SEQ ID NO: 20 or an amino acid sequence, wherein one or a few amino acids have been deleted, substituted, or added in the amino acid sequence of SEQ ID NO: 20, as an amino acid sequence of light chain variable region;
(6) an antibody recognizing a non-reducing mannose residue, comprising
the amino acid sequence of SEQ ID NO: 22 or an amino acid sequence, wherein one or a few amino acids have been deleted, substituted, or added in the amino acid sequence of SEQ ID NO: 22, as an amino acid sequence of heavy chain variable region, and the amino acid sequence of SEQ ID NO: 24 or an amino acid sequence, wherein one or a few amino acids have been deleted, substituted, or added in the amino acid sequence of SEQ ID NO: 24, as an amino acid sequence of light chain variable region;
(7) an antibody recognizing a non-reducing mannose residue, comprising
the amino acid sequence of SEQ ID NO: 26 or an amino acid sequence, wherein one or a few amino acids have been deleted, substituted, or added in the amino acid sequence of SEQ ID NO: 26, as an amino acid sequence of heavy chain variable region, and the amino acid sequence of SEQ ID NO: 28 or an amino acid sequence, wherein one or a few amino acids have been deleted, substituted, or added in the amino acid sequence of SEQ ID NO: 28, as an amino acid sequence of light chain variable region;
(8) an antibody recognizing a non-reducing mannose residue, comprising
the amino acid sequence of SEQ ID NO: 30 or an amino acid sequence, wherein one or a few amino acids have been deleted, substituted, or added in the amino acid sequence of SEQ ID NO: 30, as an amino acid sequence of heavy chain variable region, and the amino acid sequence of SEQ ID NO: 32 or an amino acid sequence, wherein one or a few amino acids have been deleted, substituted, or added in the amino acid sequence of SEQ ID NO: 32, as an amino acid sequence of light chain variable region;
(9) an antibody recognizing a non-reducing mannose residue, comprising
the amino acid sequence of SEQ ID NO: 34 or an amino acid sequence, wherein one or a few amino acids have been deleted, substituted, or added in the amino acid sequence of SEQ ID NO: 34, as an amino acid sequence of heavy chain variable region, and the amino acid sequence of SEQ ID NO: 36 or an amino acid sequence, wherein one or a few amino acids have been deleted, substituted, or added in the amino acid sequence of SEQ ID NO: 36, as an amino acid sequence of light chain variable region;
(10) an antibody recognizing a non-reducing mannose residue, comprising
the amino acid sequence of SEQ ID NO: 38 or an amino acid sequence, wherein one or a few amino acids have been deleted, substituted, or added in the amino acid sequence of SEQ ID NO: 38, as an amino acid sequence of heavy chain variable region, and the amino acid sequence of SEQ ID NO: 40 or an amino acid sequence, wherein one or a few amino acids have been deleted, substituted, or added in the amino acid sequence of SEQ ID: 40, as an amino acid sequence of light chain variable region;
(11) an antibody recognizing a non-reducing mannose residue, comprising
the amino acid sequence of SEQ ID NO: 42 or an amino acid sequence, wherein one or a few amino acids have been deleted, substituted, or added in the amino acid sequence of SEQ ID NO: 42, as an amino acid sequence of heavy chain variable region, and the amino acid sequence of SEQ ID NO: 44 or an amino acid sequence, wherein one or a few amino acids have been deleted, substituted, or added in the amino acid sequence of SEQ ID: 44, as an amino acid sequence of light chain variable region; and
(12) an antibody recognizing a non-reducing mannose residue, comprising
the amino acid sequence of SEQ ID NO: 46 or an amino acid sequence, wherein one or a few amino acids have been deleted, substituted, or added in the amino acid sequence of SEQ ID NO: 46, as an amino acid sequence of heavy chain variable region, and the amino acid sequence of SEQ ID NO: 48 or an amino acid sequence, wherein one or a few amino acids have been deleted, substituted, or added in the amino acid sequence of SEQ ID NO: 48, as an amino acid sequence of light chain variable region.

2. A nucleic acid encoding the antibody of claim 1.

3. A nucleic acid of any of the following:
(1) a nucleic acid comprising the nucleotide sequence of SEQ ID NO: 1 and the nucleotide sequence of SEQ ID NO: 3;
(2) a nucleic acid comprising the nucleotide sequence of SEQ ID NO: 5 and the nucleotide sequence of SEQ ID NO: 7;
(3) a nucleic acid comprising the nucleotide sequence of SEQ ID NO: 9 and the nucleotide sequence of SEQ ID NO: 11;
(4) a nucleic acid comprising the nucleotide sequence of SEQ ID NO: 13 and the nucleotide sequence of SEQ ID NO: 15;
(5) a nucleic acid comprising the nucleotide sequence of SEQ ID NO: 17 and the nucleotide sequence of SEQ ID NO: 19;
(6) a nucleic acid comprising the nucleotide sequence of SEQ ID NO: 21 and the nucleotide sequence of SEQ ID NO: 23;
(7) a nucleic acid comprising the nucleotide sequence of SEQ ID NO: 25 and the nucleotide sequence of SEQ ID NO: 27;
(8) a nucleic acid comprising the nucleotide sequence of SEQ ID NO: 29 and the nucleotide sequence of SEQ ID NO: 31;
(9) a nucleic acid comprising the nucleotide sequence of SEQ ID NO: 33 and the nucleotide sequence of SEQ ID NO: 35;
(10) a nucleic acid comprising the nucleotide sequence of SEQ ID NO: 37 and the nucleotide sequence of SEQ ID NO: 39;
(11) a nucleic acid comprising the nucleotide sequence of SEQ ID NO: 41 and the nucleotide sequence of SEQ ID NO: 43; and
(12) a nucleic acid comprising the nucleotide sequence of SEQ ID NO: 45 and the nucleotide sequence of SEQ ID NO: 47.

4. A process for producing an antibody wherein the nucleic acid of claim 3 is used.

5. A recombinant antibody recognizing a nonreducing mannose residue which can be obtained by the process of claim 4.

6. The recombinant antibody of claim 5 wherein the antibody is a single chain antibody.
